SEQUENCE LISTING (33 sequences)

SEQ ID NO: 1 - DNA, 146 bp, synthetic construct
  misc_feature 1..146: note = synthetic HNF1-shortABP-SynO-TSS
  source 1..146: mol_type = other DNA
gttaatcattaagtcgttaatttttgtggcccttgcgatgtttgctctggttaataatctcaggacaaacagaggttaataattttccagatctctctgagcaatagtataaaaggccagcagcagcctgaccacatctcatcctc

SEQ ID NO: 2 - DNA, 159 bp, synthetic construct
  misc_feature 1..159: note = synthetic ABPshort-HP1-AFP-TSS
  source 1..159: mol_type = other DNA
gttaatttttgtggcccttgcgatgtttgctctggttaataatctcaggacaaacatacattttcagtcatatgtttgctcactgaaggttactagttaacaggcatcccttaaacaggatataaaaggccagcagcagcctgaccacatctcatcctc

SEQ ID NO: 3 - DNA, 152 bp, synthetic construct
  misc_feature 1..152: note = HNF1-ABP-SynO
  source 1..152: mol_type = other DNA
gttaatcattaagtcgttaatttttaaaaagcagtcaaaagtccaagtggcccttgcgagcatttactctctctgtttgctctggttaataatctcaggagcacaaacagaggttaataattttccagatctctctgagcaatagtataaaa

SEQ ID NO: 4 - DNA, 150 bp, synthetic construct
  misc_feature 1..150: note = synthetic SP1-ABP-SynO
  source 1..150: mol_type = other DNA
tgggcggagtgtcgttaatttttaaaaagcagtcaaaagtccaagtggcccttgcgagcatttactctctctgtttgctctggttaataatctcaggagcacaaacagaggttaataattttccagatctctctgagcaatagtataaaa

SEQ ID NO: 5 - DNA, 137 bp, synthetic construct
  misc_feature 1..137: note = synthetic ABP-SynO
  source 1..137: mol_type = other DNA
gttaatttttaaaaagcagtcaaaagtccaagtggcccttgcgagcatttactctctctgtttgctctggttaataatctcaggagcacaaacagaggttaataattttccagatctctctgagcaatagtataaaa

SEQ ID NO: 6 - DNA, 172 bp, synthetic construct
  misc_feature 1..172: note = synthetic ABP-HP1-AFP
  source 1..172: mol_type = other DNA
gttaatttttaaaaagcagtcaaaagtccaagtggcccttgcgagcatttactctctctgtttgctctggttaataatctcaggagcacaaacagaggttaataattttcagtcatatgtttgctcactgaaggttactagttaacaggcatcccttaaacaggatataaaa

SEQ ID NO: 7 - DNA, 4377 bp, synthetic construct
  misc_feature 1..4377: note = synthetic hcoAn53
  source 1..4377: mol_type = other DNA
atgcagattgagctgtccacttgctttttcctgtgcctgctgcagttttcattttccgccactagaagatactacctgggggctgtcgaactgtcctgggattacatgcagtccgacctgctgtctgagctgcatgtggacacccgatttccacctcgcgtcccacgaagcttcccctttaatacatccgtgatgtacaagaaaactgtgttcgtcgagttcaccgatcacctgttcaacatcgcaaagccccggccaccctggatgggactgctgggccctaccatcagagccgaggtgtacgacaccgtggtcattacactgaaaaacatggcaagtcaccccgtgtcactgcatgccgtgggagtctcctactggaaggcatctgaaggcgccgagtatgacgatcagactagtcagagagaaaaagaggacgataaggtgtttcccggagaatctcatacctatgtgtggcaggtcctgaaggagaatggccctatggccagcgaccctccatgcctgacctactcctatctgtctcacgtggacctggtcaaagatctgaactccgggctgatcggagccctgctggtgtgtcgggaaggatctctggctaaggagagaacccagacactgcatcagttcgtgctgctgttcgctgtctttgacgaaggcaaaagttggcactcagagacaaaggattccctgactcaggcaatggactctgccagtgctagggcatggccaaaaatgcacaccgtgaacggctacgtcaatagaagcctgccaggactgatcggatgccacaggaagtccgtgtattggcatgtcatcggcatggggaccacaccagaagtccactctattttcctggagggacatacatttctggtgcggaatcacagacaggctagcctggagatctcccccattaccttcctgacagcacagactctgctgatggatctgggccagttcctgctgttttgccacatcagctcccaccagcatgatgggatggaggcctacgtgaaagtcgacagctgtccagaggaaccccagctgaggatgaagaacaatgaggaagaggaagactacgacgatgacctgtatgacagcgagatggatgtggtccgattcgatgacgataactcacccccttttatccagattagaagcgtcgccaagaaacaccctaagacttgggtgcattacatcgccgctgaggaagaggactgggattatgctccttccgtgctgaccccagacgatcgcagctacaaatcccagtatctgaacaatggccctcagaggattgggcgcaagtacaagaaagtgaggttcatggcttataccgatgaaaccttcaagactcgcgaagcaatccagtacgagtccggaattctgggcccactgctgtatggggaagtgggagacaccctgctgatcattttcaagaaccaggcctctaggccctacaatatctatcctcatggcattacagatgtgtctcccctgcacagtggacgcctgcctaagggcgtgaaacacctgaaggacctgcctatcctgccaggggaaatttttaagtacaaatggactgtgaccgtcgaggatggaccaactaagagcgaccccaggtgcctgacccgctactattctagtttcatcaatctggaaagagatctggcaagcggactgatcggaccactgctgatttgttacaaagagtccgtggatcagcgaggcaaccagatgatgtctgacaagcggaatgtgatcctgttctcagtctttgacgaaaaccgcagctggtatctgaccgagaacatgcagcgattcctgccaaatgcagcaggagtgcagccacaggatcctgagtttcaggctagtaacatcatgcattcaattaatggctacgtgttcgactcactgcagctgagcgtgtgtctgcacgaggtcgcttactggtatatcctgagcgtcggagcacagacagatttcctgtccgtgttcttttctggctacacttttaagcataaaatggtgtatgaggacacactgactctgttccctttttccggcgaaaccgtctttatgtctatggagaatccagggctgtgggtgctgggatgccacaactccgatttccggaatagaggaatgactgccctgctgaaagtgtcaagctgtgaccggaacaccggcgactactatgaagatacatacgaggacatcccaacttatctgctgtctgaaaacaatgtgattgagcccagaagcttcagccagaatccacccgtgctgaagcgacaccagcgggaaatcaccctgactaccctgcagtcagagcaggaagagattgattacgacgataccatcagcattgaaacaaaaagggaggacttcgatatctatggggaagacgagaaccagggacctcgctccttccagaagaggacacgccattactttattgctgcagtggagaggctgtgggattatgggatgtcccgctctccccacgtcctgcgaaatcgggcccagagtggatcagtgcctcagttcaagaaagtggtcttccaggagtttactgacgggagctttacccagcctctgtaccggggagaactgaacgagcacctgggactgctgggcccatatatcagagcagaagtggaggataacattatggtcaccttcaagaatcaggccagtcggccctactcattttattcctctctgatcagctacgaagaggaccagcgccagggcgcagaaccacgaaaaaacttcgtgaagcccaatgagaccaaaacatacttttggaaggtgcagcaccatatggctcctacaaaagacgaattcgattgcaaggcctgggcttattttagtgacgtggatctggagaaggacatgcactcagggctgatcggacctctgctgatttgtcatactaacaccctgaatccagcacacggacgacaggtgacagtccaggaattcgctctgttctttacaatcttcgatgagactaagagctggtacttcactgaaaacatggagagaaattgcagggccccttgtaatatccagatggaagacccaacattcaaggagaactacagatttcatgctattaatggctatgtgatggatactctgccagggctggtcatggcacaggaccagagaatcaggtggtacctgctgtctatggggagtaacgagaatatccacagcattcatttctccggacacgtgtttactgtcaggaagaaagaagagtataaaatggccgtgtacaacctgtatccaggcgtgttcgaaaccgtcgagatgctgccaagcaaggcaggaatctggcgagtggaatgcctgattggcgagcacctgcatgctgggatgagtaccctgtttctggtgtactcaaaacagtgtcagacacctctgggaatggcatctggccatatccgggatttccagattaccgcaagtggacagtacggacagtgggctccaaagctggcaagactgcactatagcggctccatcaacgcctggtctacaaaagagccctttagttggattaaggtggacctgctggcccctatgatcattcatggcatcaaaactcagggggctaggcagaagttcagttcactgtacatcagccagtttatcatcatgtactccctggatgggaagaaatggcagacctaccgcgggaatagcacaggaactctgatggtgttctttggaaacgtcgacagctccggcatcaagcacaacattttcaatcctccaatcattgcccgctacatccgactgcaccccacccattattcaattcgaagcacactgcggatggaactgatgggctgcgatctgaactcttgtagtatgcctctggggatggagtctaaggccatcagtgacgctcagattaccgcatctagttacttcaccaatatgtttgccacatggtcaccaagccaggctaggctgcacctgcagggaagaacaaacgcctggaggcctcaggtgaacaatccaaaggagtggctgcaggtggatttccagaaaactatgaaggtcaccggaatcacaactcagggcgtgaaatcactgctgaccagcatgtatgtgaaggagtttctgatttcaagctcccaggacggccaccattggacactgttcctgcagaacgggaaggtgaaagtcttccagggaaatcaggattcttttacaccagtggtcaacagtctggacccccctctgctgactcggtacctgagaatccacccccagagctgggtccatcagattgcactgcgactggaagtgctgggatgcgaggcacagcagctgtattga

SEQ ID NO: 8 - DNA, 4359 bp, synthetic construct
  misc_feature 1..4359: note = synthetic lcoAn53
  source 1..4359: mol_type = other DNA
atgcagattgagctgagcacctgcttcttcctgtgcctgctgcagttctcattctctgccaccaggagatactacctgggcgccgtggagctgagctgggactacatgcagtctgacctgctgtctgagctgcatgtggacaccaggttcccccccagagtgccccgaagcttccccttcaacaccagcgtgatgtacaagaagaccgtgttcgtggagttcactgaccacctgttcaacatcgccaagcccaggcccccctggatgggcctgctgggccccaccatcagagccgaggtgtacgacaccgtggtcatcaccctgaagaacatggccagccaccccgtctccctgcacgccgtgggggtgagctactggaaggcctctgagggcgccgagtacgacgaccagaccagccagagggagaaggaggacgacaaggtgttccctggggaaagccacacctacgtgtggcaggtcctgaaggagaacggccccatggcctctgaccccccatgcctgacctacagctacctgagccacgtggacctggtgaaggacctgaactctggcctgattggggccctgctggtgtgcagggagggcagcctggccaaggagagaacccagaccctgcaccagttcgtgctgctgttcgccgtgttcgacgagggcaagagctggcactctgaaaccaaggatagcctgactcaggccatggactctgcctctgccagggcctggcccaagatgcacaccgtcaacggctacgtcaacaggagcctgcctggcctgattggctgccacaggaagagcgtgtactggcatgtgatcggcatgggcaccacccctgaggtgcacagcatcttcctggagggccacaccttcctggtcaggaaccacaggcaggccagcctggagatcagccccatcaccttcctgaccgcccagaccctgctgatggacctgggccagttcctgctgttctgccacatctccagccaccagcacgacggcatggaggcctacgtgaaagtggacagctgccctgaggagccccagctgaggatgaagaacaacgaggaggaggaggactatgatgacgacctgtatgacagcgagatggacgtggtcaggttcgacgacgacaacagcccccctttcatccagatcaggagcgtggccaagaagcaccccaagacctgggtgcactacatcgctgctgaggaggaggactgggactatgccccctccgtgctgacccctgatgacaggagctacaagagccagtacctgaacaatggcccccagaggattggcaggaagtacaagaaagtcaggttcatggcctacactgatgaaaccttcaagaccagggaggccatccagtacgagtctggcatcctgggccccctgctgtacggggaggtgggggacaccctgctgatcatcttcaagaaccaggccagcaggccctacaacatctacccccatggcatcaccgacgtgagccccctgcacagcggaaggctgcctaagggggtgaagcacctgaaagacctgcccatcctgcctggggagatcttcaagtacaagtggactgtgactgtggaggacggccccaccaagagcgaccccaggtgcctgaccagatactacagcagcttcatcaacctggagagggacctggcctctggcctgattggccccctgctgatctgctacaaggagtctgtggaccagaggggcaaccagatgatgagcgacaagaggaacgtgatcctgttctctgtcttcgacgagaacaggagctggtacctgaccgagaacatgcagaggttcctgcccaacgcagctggggtgcagccacaggaccccgagttccaggccagcaacatcatgcacagcatcaatggctacgtgttcgacagcctgcagctgagcgtgtgcctgcacgaggtggcctactggtacatcctgagcgtcggcgcccagaccgacttcctgagcgtgttcttctctggctacaccttcaagcacaagatggtgtatgaggacaccctgaccctgttccccttcatgagcatggagaaccctggcctgtgggtgctgggctgccacaacagcgacttcaggaacaggggcatgactgccctgctgaaagtctccagctgtgaccggaacaccggggactactacgaggacacatacgaggacatcccaacttacctgctgagcgaaaacaatgtgatcgagcccaggagcttctctcagaaccccccagtgctgaagaggcaccagagggagatcaccctgaccaccctgcagtctgagcaggaggagatcgactatgatgacaccatcagcattgagacaaagagggaggacttcgacatctacggggaggacgagaaccagggacccaggagcttccagaagaggaccaggcactacttcattgctgctgtggagaggctgtgggactatggcatgtcccgcagcccccatgtgctgaggaacagggcccagtctggcagcgtgccccagttcaagaaagtcgtgttccaggagttcaccgacggcagcttcacccagcccctgtacagaggggagctgaacgagcacctgggcctgctgggcccctacatcagggccgaggtggaggacaacatcatggtgaccttcaagaaccaggccagcaggccctacagcttctacagcagcctgatcagctacgaggaggaccagaggcagggggctgagcccaggaagaactttgtgaagcccaatgaaaccaagacctacttctggaaggtgcagcaccacatggcccccaccaaggacgagttcgactgcaaggcctgggcctacttctctgacgtggacctggagaaggacatgcactctggcctgattggccccctgctgatttgccacaccaacaccctgaaccctgcccatggcaggcaggtgactgtgcaggagttcgccctgttcttcaccatcttcgatgaaaccaagagctggtacttcactgagaacatggagaggaactgcagggccccctgcaacatccagatggaggaccccaccttcaaggagaactacaggttccatgccatcaatggctacgtgatggacaccctgcctggcctggtcatggcccaggaccagaggatcaggtggtatctgctgagcatgggcagcaacgagaacatccacagcatccacttctctggccacgtgttcactgtgaggaagaaggaggagtacaagatggccgtgtacaacctgtaccctggggtgttcgaaaccgtggagatgctgcccagcaaggccggcatctggagggtggagtgcctgattggggagcacctgcacgccggcatgagcaccctgttcctggtgtacagcaaacagtgccagacccccctgggcatggcctctggccacatcagggacttccagatcactgcctctggccagtacggccagtgggcccccaagctggccaggctgcactactccggaagcatcaatgcctggagcaccaaggagcccttcagctggatcaaagtggacctgctggcccccatgatcatccacggcatcaagacccagggggccaggcagaagttctccagcctgtacatcagccagttcatcatcatgtacagcctggacggcaagaagtggcagacctacaggggcaacagcaccggcaccctgatggtgttcttcggcaacgtggacagcagcggcatcaagcacaacatcttcaacccccccatcatcgccagatacatcaggctgcaccccacccactacagcatcaggagcaccctgaggatggagctgatgggctgtgacctgaacagctgcagcatgcccctgggcatggagagcaaggccatctctgacgcccagatcactgcctccagctacttcaccaacatgtttgccacctggagccccagccaggccaggctgcacctgcagggcaggacaaatgcctggaggccccaggtcaacaaccccaaggagtggctgcaggtggacttccagaagaccatgaaggtgactgggatcaccacccagggggtgaagagcctgctgaccagcatgtacgtgaaggagttcctgatctccagcagccaggacggccaccattggaccctgttcctgcagaatggcaaggtgaaggtgttccagggcaaccaggacagcttcacccctgtggtcaacagcctggacccccccctgctgaccagatacctgaggatccacccccagagctgggtgcaccagatcgccctgaggctggaggtgctgggctgtgaggcccagcagctgtactga

SEQ ID NO: 9 - DNA, 4404 bp, synthetic construct
  misc_feature 1..4404: note = synthetic lcoET3 with CpGs
  source 1..4404: mol_type = other DNA
atgcagctggaactgtctacctgtgtgtttctgtgtctgctgcctctggggttttctgctatccgccgctactatctgggagccgtggagctgtcctgggactacaggcagagcgagctgctgagagaactgcacgtggataccagattcccagctaccgctccaggagctctgcctctgggcccatccgtgctgtacaagaaaaccgtcttcgtggagtttaccgaccagctgttcagcgtggccaggccaagaccaccttggatgggactgctgggaccaaccatccaggctgaggtgtacgataccgtggtcgtgaccctgaaaaacatggcctcccatcccgtgagcctgcacgctgtcggggtgtccttctggaagtccagcgagggagccgagtacgaagaccatacctcccagcgcgagaaagaagacgataaggtgctgcctggcaaaagccagacctatgtctggcaggtgctgaaggagaacggaccaaccgctagcgacccaccatgcctgacctactcttatctgtcccacgtcgatctggtgaaggacctgaattccggactgatcggagctctgctggtgtgtagagagggaagcctgaccagagaaagaacccagaacctgcatgagttcgtcctgctgttcgccgtgtttgacgaagggaagagctggcactctgcccgcaatgactcctggaccagagctatggatccagctcctgctagagctcagcctgctatgcacaccgtcaacggctacgtgaatcggtctctgccaggactgatcggctgccataagaaaagcgtctattggcacgtgatcggaatgggcaccagccccgaggtgcattctatcttcctggaaggccacacctttctggtcaggcaccatagacaggcctctctggagatctcccctctgaccttcctgaccgctcagacctttctgatggacctggggcagttcctgctgttttgccatatctcttcccaccatcacggaggaatggaggctcacgtcagggtggaatcctgtgctgaggaaccacagctgagaagaaaggctgatgaggaagaggactacgacgataacctgtatgacagcgatatggacgtcgtgcgcctggacggcgacgatgtcagccctttcatccagatccggtctgtggccaagaaacatccaaagacctgggtccactacatcgccgctgaagaggaagattgggactatgcccccctggtgctggctcctgacgatagatcctacaaaagccagtatctgaacaatgggccccagcgcatcggacggaagtacaagaaagtgaggttcatggcctataccgacgagacctttaagaccagagaggctatccagcacgaatccgggatcctgggacctctgctgtacggcgaagtgggggataccctgctgatcatcttcaagaaccaggcctccaggccatacaatatctatccccatggcatcaccgacgtgagaccactgtacagcaggagactgcccaagggggtcaaacacctgaaggatttccccatcctgcctggagagatctttaagtataaatggaccgtcaccgtggaagacgggcctaccaagtccgatccacgctgcctgacccggtactatagctctttcgtgaacatggagagagacctggctagcggactgatcggacccctgctgatctgttacaaagagagcgtggaccagaggggcaaccagatcatgtctgataagagaaatgtcatcctgttctccgtgtttgacgagaaccgcagctggtacctgaccgagaacatccagcggttcctgccaaatccagctggagtgcagctggaggacccagaatttcaggcttccaacatcatgcatagcatcaatggctacgtgttcgatagcctgcagctgtctgtctgcctgcacgaggtggcctactggtatatcctgtccatcggcgctcagaccgacttcctgtccgtgttctttagcgggtacacctttaagcataaaatggtgtatgaggataccctgaccctgttccccttttctggcgagaccgtgttcatgtccatggaaaaccctggcctgtggatcctggggtgccacaacagcgacttcaggaatagaggaatgaccgccctgctgaaagtgtccagctgtgataagaataccggcgattactatgaggactcttacgaagatatctccgcttatctgctgagcaagaacaatgccatcgagcccaggtctttcgctcagaactccagacctccaagcgcttctgctcctaagccacctgtgctgagaagacatcagagggacatctccctgcctaccttccagccagaggaagataaaatggactacgacgatatcttcagcaccgagaccaagggggaagattttgacatctatggagaggacgaaaaccaggatccaagatccttccagaagagaaccagacactactttatcgccgctgtggagcagctgtgggactatgggatgtccgaaagcccacgggccctgaggaacagagctcagaatggagaggtgccccgcttcaagaaagtcgtgttccgggagtttgccgacggcagctttacccagccatcttacaggggggagctgaacaagcatctggggctgctgggaccctatatcagagccgaggtcgaagataacatcatggtgaccttcaagaatcaggcttctcgcccctactccttttattcttccctgatctcctaccctgacgatcaggagcagggcgccgaacctaggcacaacttcgtgcagccaaatgagaccagaacctacttttggaaggtgcagcatcacatggctcccaccgaggatgaattcgactgcaaagcttgggcctatttttccgatgtcgacctggagaaggacgtgcatagcggcctgatcgggcctctgctgatctgtcgcgccaacaccctgaatgctgctcacggaagacaggtcaccgtgcaggagttcgctctgttctttaccatctttgacgaaaccaagagctggtacttcaccgagaacgtggaaaggaattgcagagccccctgtcatctgcagatggaggaccctaccctgaaggaaaactacaggttccacgccatcaatggatatgtcatggataccctgcccggcctggtcatggctcagaaccagcgcatccggtggtacctgctgtctatgggatccaacgagaatatccatagcatccacttctctggccatgtcttttccgtgaggaagaaagaggaatacaaaatggccgtgtacaatctgtatcctggggtcttcgagaccgtggaaatgctgccaagcaaagtgggaatctggagaatcgagtgcctgatcggcgaacacctgcaggccgggatgagcaccaccttcctggtgtactctaagaaatgtcagaccccactggggatggcctccggacatatccgcgacttccagatcaccgctagcggacagtacggacagtgggctccaaagctggctagactgcactattctggctccatcaacgcctggtctaccaaagagccattctcctggatcaaggtggacctgctggcccccatgatcatccacggaatcaaaacccagggcgctaggcagaagttcagctctctgtacatctcccagtttatcatcatgtatagcctggacgggaagaaatggcagacctacagaggcaattccaccgggaccctgatggtcttctttggaaacgtggattccagcggcatcaagcacaacatcttcaatccacccatcatcgcccgctacatccggctgcatcctacccactatagcatcaggtctaccctgagaatggagctgatgggatgcgacctgaacagctgttctatgccactgggcatggagtccaaggctatcagcgatgcccagatcaccgcttcttcctacttcaccaatatgtttgctacctggtccccaagcaaggctagactgcacctgcagggaagatccaacgcttggagaccccaggtgaacaatcctaaggagtggctgcaggtcgacttccagaaaaccatgaaggtcaccggggtgaccacccagggagtgaaatctctgctgacctccatgtacgtcaaggagttcctgatcagctcttcccaggacggccaccagtggaccctgttctttcagaacggcaaggtcaaagtgttccaggggaatcaggactcttttacccccgtcgtgaactccctggatcctccactgctgaccaggtacctgagaatccatcctcagagctgggtgcaccagatcgctctgagaatggaggtcctgggatgcgaagctcaggacctgtattga

SEQ ID NO: 10 - DNA, 4403 bp, synthetic construct
  misc_feature 1..4403: note = synthetic lcoET3 CpGs removed
  source 1..4403: mol_type = other DNA
atgcagctggaactgtctacctgtgtgtttctgtgtctgctgcctctggggttttctgctatcaggagatactatctgggagctgtggagctgtcctgggactacaggcagtctgagctgctgagagaactgcatgtggataccagattcccagctacagctccaggagctctgcctctgggcccatctgtgctgtacaagaaaacagtctttgtggagtttacagaccagctgttctctgtggccaggccaagaccaccttggatgggactgctgggaccaaccatccaggctgaggtgtatgatacagtggtggtgaccctgaaaaacatggcctcccatcctgtgagcctgcatgctgtgggggtgtccttctggaagtcctctgagggagctgagtatgaagaccatacctcccagagggagaaagaagatgataaggtgctgcctggcaaaagccagacctatgtctggcaggtgctgaaggagaatggaccaactgcttctgacccaccatgcctgacctactcttatctgtcccatgtggatctggtgaaggacctgaattctggactgattggagctctgctggtgtgtagagagggaagcctgaccagagaaagaacccagaacctgcatgagtttgtcctgctgtttgctgtgtttgatgaagggaagagctggcactctgccaggaatgactcctggaccagagctatggatccagctcctgctagagctcagcctgctatgcacacagtcaatggctatgtgaataggtctctgccaggactgattggctgccataagaaatctgtctattggcatgtgattggaatgggcaccagccctgaggtgcattctatcttcctggaaggccacacctttctggtcaggcaccatagacaggcctctctggagatctcccctctgaccttcctgacagctcagacctttctgatggacctggggcagttcctgctgttttgccatatctcttcccaccatcatggaggaatggaggctcatgtcagggtggaatcctgtgctgaggaaccacagctgagaagaaaggctgatgaggaagaggactatgatgataacctgtatgactctgatatggatgtggtgaggctggatggggatgatgtcagccctttcatccagatcaggtctgtggccaagaaacatccaaagacctgggtccactacattgctgctgaagaggaagattgggactatgcccccctggtgctggctcctgatgatagatcctacaaaagccagtatctgaacaatgggccccagaggattggaaggaagtacaagaaagtgaggttcatggcctatacagatgagacctttaagaccagagaggctatccagcatgaatctgggatcctgggacctctgctgtatggagaagtgggggatacctgctgatcatcttcaagaaccaggcctccaggccatacaatatctatccccatggcatcacagatgtgagaccactgtacagcaggagactgcccaagggggtcaaacacctgaaggatttccccatcctgcctggagagatctttaagtataaatggacagtcacagtggaagatgggcctaccaagtctgatccaaggtgcctgaccagatactatagctcttttgtgaacatggagagagacctggcttctggactgattggacccctgctgatctgttacaaagagtctgtggaccagaggggcaaccagatcatgtctgataagagaaatgtcatcctgttctctgtgtttgatgagaacaggagctggtacctgacagagaacatccagaggttcctgccaaatccagctggagtgcagctggaggacccagaatttcaggcttccaacatcatgcatagcatcaatggctatgtgtttgatagcctgcagctgtctgtctgcctgcatgaggtggcctactggtatatcctgtccattggagctcagacagacttcctgtctgtgttctttagtgggtacacctttaagcataaaatggtgtatgaggataccctgaccctgttccccttttctggggagacagtgttcatgtccatggaaaaccctggcctgtggatcctggggtgccacaactctgacttcaggaatagaggaatgacagccctgctgaaagtgtccagctgtgataagaatacaggggattactatgaggactcttatgaagatatctctgcttatctgctgagcaagaacaatgccattgagcccaggtcttttgctcagaactccagacctccatctgcttctgctcctaagccacctgtgctgagaagacatcagagggacatctccctgcctaccttccagccagaggaagataaaatggactatgatgatatcttcagcacagagaccaagggggaagattttgacatctatggagaggatgaaaaccaggatccaagatccttccagaagagaaccagacactactttattgctgctgtggagcagctgtgggactatgggatgtctgaaagcccaagggccctgaggaacagagctcagaatggagaggtgcccagattcaagaaagtggtgttcagagagtttgctgatggcagctttacccagccatcttacaggggggagctgaacaagcatctggggctgctgggaccctatatcagagctgaggtggaagataacatcatggtgaccttcaagaatcaggcttctaggccctactccttttattcttccctgatctcctaccctgatgatcaggagcagggagctgaacctaggcacaactttgtgcagccaaatgagaccagaacctacttttggaaggtgcagcatcacatggctcccacagaggatgaatttgactgcaaagcttgggcctatttttctgatgtggacctggagaaggatgtgcattctggcctgattgggcctctgctgatctgtagggccaacaccctgaatgctgctcatggaagacaggtcacagtgcaggagtttgctctgttctttaccatctttgatgaaaccaagagctggtacttcacagagaatgtggaaaggaattgcagagccccctgtcatctgcagatggaggaccctaccctgaaggaaaactacaggttccatgccatcaatggatatgtcatggataccctgcctggcctggtcatggctcagaaccagaggatcagatggtacctgctgtctatgggatccaatgagaatatccatagcatccacttctctggccatgtcttttctgtgaggaagaaagaggaatacaaaatggctgtgtacaatctgtatcctggggtctttgagacagtggaaatgctgccaagcaaagtgggaatctggagaattgagtgcctgattggggaacacctgcaggctgggatgagcaccaccttcctggtgtactctaagaaatgtcagaccccactggggatggcctctggacatatcagggacttccagatcacagcttctggacagtatggacagtgggctccaaagctggctagactgcactattctggctccatcaatgcctggtctaccaaagagccattctcctggatcaaggtggacctgctggcccccatgatcatccatggaatcaaaacccagggagctaggcagaagttcagctctctgtacatctcccagtttatcatcatgtatagcctggatgggaagaaatggcagacctacagaggcaattccactgggaccctgatggtcttctttggaaatgtggattcctctggcatcaagcacaacatcttcaatccacccatcattgccaggtacatcaggctgcatcctacccactatagcatcaggtctaccctgagaatggagctgatgggatgtgacctgaacagctgttctatgccactgggcatggagtccaaggctatctctgatgcccagatcacagcttcttcctacttcaccaatatgtttgctacctggtccccaagcaaggctagactgcacctgcagggaagatccaatgcttggagaccccaggtgaacaatcctaaggagtggctgcaggtggacttccagaaaaccatgaaggtcacaggggtgaccacccagggagtgaaatctctgctgacctccatgtatgtcaaggagttcctgatcagctcttcccaggatggccaccagtggaccctgttctttcagaatggcaaggtcaaagtgttccaggggaatcaggactcttttaccccagtggtgaactccctggatcctccactgctgaccaggtacctgagaatccatcctcagagctgggtgcaccagattgctctgagaatggaggtcctgggatgtgaagctcaggacctgtattga

SEQ ID NO: 11 - DNA, 4404 bp, synthetic construct
  misc_feature 1..4404: note = synthetic NoCoET3
  source 1..4404: mol_type = other DNA
atgcagctagagctctccacctgtgtctttctgtgtctcttgccactcggctttagtgccatcaggagatactacctgggcgcagtggaactgtcctgggactaccggcaaagtgaactcctccgtgagctgcacgtggacaccagatttcctgctacagcgccaggagctcttccgttgggcccgtcagtcctgtacaaaaagactgtgttcgtagagttcacggatcaacttttcagcgttgccaggcccaggccaccatggatgggtctgctgggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtcttacctactcatacctgtctcacgtggacctggtgaaagacctgaattcgggcctcattggagccctgctggtttgtagagaagggagtctgaccagagaaaggacccagaacctgcacgaatttgtactactttttgctgtctttgatgaagggaaaagttggcactcagcaagaaatgactcctggacacgggccatggatcccgcacctgccagggcccagcctgcaatgcacacagtcaatggctatgtcaacaggtctctgccaggtctgatcggatgtcataagaaatcagtctactggcacgtgattggaatgggcaccagcccggaagtgcactccatttttcttgaaggccacacgtttctcgtgaggcaccatcgccaggcttccttggagatctcgccactaactttcctcactgctcagacattcctgatggaccttggccagttcctactgttttgtcatatctcttcccaccaccatggtggcatggaggctcacgtcagagtagaaagctgcgccgaggagccccagctgcggaggaaagctgatgaagaggaagattatgatgacaatttgtacgactcggacatggacgtggtccggctcgatggtgacgacgtgtctccctttatccaaatccgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagacgcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatccgcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaacctaggagctttgcccagaattcaagaccccctagtgcgagcgctccaaagcctccggtcctgcgacggcatcagagggacataagccttcctacttttcagccggaggaagacaaaatggactatgatgatatcttctcaactgaaacgaagggagaagattttgacatttacggtgaggatgaaaatcaggaccctcgcagctttcagaagagaacccgacactatttcattgctgcggtggagcagctctgggattacgggatgagcgaatccccccgggcgctaagaaacagggctcagaacggagaggtgcctcggttcaagaaggtggtcttccgggaatttgctgacggctccttcacgcagccgtcgtaccgcggggaactcaacaaacacttggggctcttgggaccctacatcagagcggaagttgaagacaacatcatggtaactttcaaaaaccaggcgtctcgtccctattccttctactcgagccttatttcttatccggatgatcaggagcaaggggcagaacctcgacacaacttcgtccagccaaatgaaaccagaacttacttttggaaagtgcagcatcacatggcacccacagaagacgagtttgactgcaaagcctgggcctacttttctgatgttgacctggaaaaagatgtgcactcaggcttgatcggcccccttctgatctgccgcgccaacaccctgaacgctgctcacggtagacaagtgaccgtgcaagaatttgctctgtttttcactatttttgatgagacaaagagctggtacttcactgaaaatgtggaaaggaactgccgggccccctgccatctgcagatggaggaccccactctgaaagaaaactatcgcttccatgcaatcaatggctatgtgatggatacactccctggcttagtaatggctcagaatcaaaggatccgatggtatctgctcagcatgggcagcaatgaaaatatccattcgattcattttagcggacacgtgttcagtgtacggaaaaaggaggagtataaaatggccgtgtacaatctctatccgggtgtctttgagacagtggaaatgctaccgtccaaagttggaatttggcgaatagaatgcctgattggcgagcacctgcaagctgggatgagcacgactttcctggtgtacagcaagaagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 12 - DNA, 4404 bp, synthetic construct
  misc_feature 1..4404: note = synthetic mcoET3
  source 1..4404: mol_type = other DNA
atgcagctggagctctcaacctgtgtgttcctctgcctgctccccctgggattttcagctatcaggagatactatctgggagcagtggaactgtcctgggactacaggcagtcagagctgctcagagaactgcatgtggatactaggttccctgcaacagctcctggagcactgccactgggaccttcagtgctgtacaagaaaactgtctttgtggagtttacagaccagctgttcagtgtggccaggcccaggcccccctggatggggctgctgggacccaccatccaggctgaagtgtatgatactgtggtggtgaccctgaaaaacatggcctctcatccagtcagcctgcatgctgtgggagtgagcttctggaagagcagtgagggagctgagtatgaagaccatacctcacagagggagaaagaagatgataaggtgctgccaggaaaaagccagacctatgtgtggcaggtgctgaaggagaatggccctacagcttcagatcctccctgcctcacatactcttatctgagccatgtggatctggtgaaggacctcaatagtggcctgattggggcactgctggtgtgcagagaggggtccctcacaagggaaagaactcagaacctgcatgagtttgtcctgctctttgctgtgtttgatgagggaaagtcctggcactcagcaaggaatgacagctggaccagggctatggacccagcaccagccagagctcagccagctatgcacactgtcaatggctatgtgaataggtccctgcctggactcattggctgccataagaaatcagtctattggcatgtgattggaatgggcaccagcccagaggtgcattccatcttcctggaaggccacacatttctggtcaggcaccatagacaggccagcctggagatcagcccactgactttcctcacagcacagacatttctgatggacctggggcagttcctgctcttttgccatatctcaagtcaccatcatggagggatggaggctcatgtcagggtggaaagctgtgcagaggaacctcagctgaggaggaaggcagatgaggaagaggactatgatgataacctgtatgactcagatatggatgtggtgaggctggatggagatgatgtcagcccattcatccagatcaggtcagtggctaagaaacaccctaagacctgggtccactacattgcagctgaagaggaagattgggactatgcacccctggtgctggccccagatgatagaagttacaaatctcagtatctgaacaatgggccccagaggattggaaggaagtacaagaaagtgaggttcatggcttatactgatgagacctttaagacaagagaggcaatccagcatgaaagtggcatcctgggaccactgctctatggagaagtgggggataccctgctcatcatcttcaagaaccaggcctcaaggccttacaatatctatccccatggcatcacagatgtgaggcctctctacagcaggagactgcccaagggagtcaaacacctcaaggatttccccatcctgccaggggaaatcttcaagtataaatggacagtcactgtggaagatgggccaactaagtcagatcctaggtgcctgaccaggtactattctagctttgtgaacatggagagggacctggcttcaggactgattggacctctgctcatctgctacaaagaatcagtggaccagaggggcaaccagatcatgagtgataagagaaatgtcatcctgttctcagtgtttgatgagaataggagttggtatctgacagaaaacatccagaggttcctgcctaatcctgcaggagtgcagctggaggacccagaatttcaggcttcaaacatcatgcatagtatcaatggctatgtgtttgatagtctgcagctctctgtctgcctgcatgaggtggcctactggtatatcctcagcattggagctcagactgacttcctgagtgtgttcttttcaggctacacattcaagcataagatggtctatgaagataccctgacactcttccccttttctggggagactgtgtttatgagcatggaaaacccaggcctgtggattctggggtgccacaacagtgacttcaggaatagagggatgactgctctgctcaaagtgtcctcatgtgataagaatactggagattactatgaggactcttatgaagatatcagtgcatatctgctctccaaaaacaatgccattgagcccaggtcatttgctcagaacagtagaccaccttctgcaagtgcaccaaagcctccagtgctgaggagacaccagagggacatcagcctgccaaccttccagcctgaggaagataaaatggactatgatgatatcttctccactgagaccaagggggaagattttgacatctatggagaggatgaaaaccaggaccccaggtccttccagaagaggaccagacactactttattgcagctgtggagcagctgtgggactatggcatgtctgaatcacctagagctctgaggaacagagcacagaatggggaggtgcccaggttcaagaaagtggtgttcagagaatttgcagatggctcttttacccagcctagctacaggggggagctcaacaagcatctggggctgctgggaccctatatcagagcagaggtggaagataacatcatggtgacattcaagaatcaggcctcaagaccctacagtttttatagttctctgatcagctacccagatgatcaggagcagggggctgaaccaaggcacaactttgtgcagcctaatgagacaagaacttacttttggaaggtccagcatcacatggctcccacagaggatgagtttgactgcaaggcctgggcatatttttctgatgtggacctggagaaggatgtgcatagtggcctcattgggccactgctcatctgcagggcaaacacactgaatgctgcacatggcaggcaggtcactgtgcaggagtttgccctgttctttacaatctttgatgaaactaagtcctggtacttcacagagaatgtggaaaggaattgcagagccccctgccatctccagatggaggacccaactctgaaggaaaactacaggttccatgctatcaatggatatgtcatggatacactgccaggcctggtgatggcacagaaccagaggatcaggtggtatctgctcagcatggggtccaatgagaatatccattctatccacttctcaggacatgtcttttcagtgaggaagaaagaggaatataaaatggctgtgtacaatctgtatccaggggtctttgagacagtggaaatgctgcctagcaaagtggggatctggagaattgagtgcctcattggagaacacctgcaggcagggatgtccaccacatttctggtgtactcaaagaaatgccagactcccctggggatggcaagtggacatatcagggacttccagatcactgcatcaggacagtatggacagtgggcaccaaagctggctaggctccactatagtggctctatcaatgcttggagtaccaaagagcctttctcttggatcaaggtggatctgctggcccccatgatcatccatggaatcaaaacacagggagctagacagaagttcagctccctgtacatcagtcagtttatcatcatgtattctctggatgggaagaaatggcagacctacaggggcaatagcactgggacactgatggtcttctttggaaatgtggattcaagtggcatcaagcacaacatcttcaatcctcccatcattgccaggtacatcagactgcatcccacacactattcaatcaggagtactctcagaatggagctgatggggtgtgacctcaacagctgctccatgccactgggaatggaatccaaggcaatctcagatgcccagatcactgcttctagctacttcaccaatatgtttgcaacatggtcacccagtaaagcaaggctgcacctccagggaaggtccaatgcttggagaccccaggtgaacaatccaaaggagtggctgcaggtggactttcagaaaaccatgaaggtcacaggggtgactacccagggagtgaaaagtctgctcacctctatgtatgtcaaggagttcctgatctcctcaagtcaggatggccaccagtggacactgttctttcagaatggcaaggtcaaagtgttccaggggaatcaggacagctttacaccagtggtgaacagcctggacccccctctgctcactagatatctgagaatccatccacagagctgggtgcaccagattgcactcagaatggaggtcctgggctgtgaagcccaggacctgtattga

SEQ ID NO: 13 - DNA, 4374 bp, synthetic construct
  misc_feature 1..4374: note = synthetic lcoHSQ with CpGs
  source 1..4374: mol_type = other DNA
atgcagatcgaactgtctacctgtttctttctgtgcctgctgcggttttgtttttccgctaccagaagatactacctgggagccgtcgaactgagctgggattacatgcagtctgacctgggagagctgcccgtggacgctagattcccacctagagtccctaagtccttccccttcaacaccagcgtggtctacaagaaaaccctgttcgtggagtttaccgaccacctgttcaacatcgctaagcctagaccaccatggatgggactgctgggaccaaccatccaggccgaggtgtacgacaccgtggtcatcaccctgaaaaacatggcttctcaccccgtgtccctgcatgctgtgggcgtctcctactggaaggccagcgaaggggctgagtatgacgatcagaccagccagcgggaaaaagaggacgataaggtgttccctggcgggtcccatacctacgtgtggcaggtcctgaaggagaatggaccaatggcttccgaccctctgtgcctgacctactcttatctgtcccacgtggacctggtcaaggatctgaacagcggcctgatcggggctctgctggtgtgtcgcgaagggtccctggccaaggagaaaacccagaccctgcataagttcatcctgctgttcgccgtgtttgacgaaggaaaaagctggcactctgagaccaagaactctctgatgcaggacagggatgccgcttccgccagagcttggcccaagatgcacaccgtgaacggctacgtcaataggagcctgcctggactgatcggctgccacagaaagtccgtgtattggcatgtcatcggaatgggcaccacccctgaagtgcacagcatcttcctggaggggcatacctttctggtccgcaaccaccggcaggctagcctggagatctctccaatcaccttcctgaccgcccagaccctgctgatggacctgggacagttcctgctgttttgccacatctccagccaccagcatgatggcatggaggcttacgtgaaagtcgactcctgtcccgaggaacctcagctgaggatgaagaacaatgaggaagccgaagactatgacgatgacctgaccgacagcgagatggatgtggtccgcttcgatgacgataactctccctcctttatccagatccggtccgtggccaagaaacaccctaagacctgggtccattacatcgccgctgaggaagaggactgggattatgctccactggtgctggcccccgacgatagatcctacaaaagccagtatctgaacaatggaccccagaggatcggcagaaagtacaagaaagtgaggttcatggcttataccgatgagacctttaagaccagagaagccatccagcacgagtccgggatcctgggacctctgctgtacggcgaagtgggggacaccctgctgatcatcttcaagaaccaggccagcaggccttacaatatctatccacatggcatcaccgatgtgagacctctgtactcccgccggctgccaaagggcgtgaaacacctgaaggacttcccaatcctgcccggggaaatctttaagtataaatggaccgtcaccgtcgaggatgggcccaccaagagcgaccctaggtgcctgaccagatactattcttccttcgtgaatatggagagagacctggcttccggactgatcggacccctgctgatctgttacaaagagagcgtggatcagcgcggcaaccagatcatgtctgacaagcggaatgtgatcctgttcagcgtctttgacgaaaaccgctcttggtacctgaccgagaacatccagcggttcctgcctaatccagctggagtgcagctggaagatcccgagttccaggcctctaacatcatgcattccatcaatggctacgtgttcgactccctgcagctgagcgtgtgcctgcacgaggtcgcttactggtatatcctgagcatcggagcccagaccgatttcctgtctgtgttcttttccggctacacctttaagcataaaatggtgtatgaggacaccctgaccctgttcccattttccggcgaaaccgtgttcatgagcatggagaatcccgggctgtggatcctgggatgccacaactccgatttcaggaatagagggatgaccgccctgctgaaagtgagctcttgtgacaagaacaccggagactactatgaagatagctacgaggacatctctgcttatctgctgtccaaaaacaatgccatcgagcccaggagcttctctcagaaccctccagtgctgaagcgccaccagcgggagatcaccagaaccaccctgcagagcgatcaggaagagatcgactacgacgataccatctccgtggaaatgaagaaagaggacttcgatatctatgacgaagatgagaaccagtctcccaggtccttccagaagaaaaccagacattactttatcgccgctgtggagcggctgtgggactatggcatgtccagctctcctcacgtgctgagaaatagagctcagtccggaagcgtcccacagttcaagaaagtggtcttccaggagtttaccgacggaagctttacccagccactgtaccgcggcgaactgaacgagcacctggggctgctgggaccctatatccgggctgaagtggaggataacatcatggtcaccttcaggaatcaggccagcagaccctactctttttattccagcctgatctcctacgaagaggaccagagacagggagctgaaccaagaaaaaacttcgtgaagcctaatgagaccaaaacctacttttggaaggtgcagcaccatatggcccctaccaaagacgagttcgattgcaaggcctgggcttattttagcgacgtggatctggagaaggacgtccactccggcctgatcgggccactgctggtgtgtcataccaacaccctgaatccagctcacggaaggcaggtgaccgtccaggaattcgccctgttctttaccatctttgatgagaccaagagctggtacttcaccgaaaacatggagaggaattgcagagccccatgtaacatccagatggaagaccccaccttcaaggagaactacagatttcatgctatcaatgggtatatcatggataccctgccaggactggtcatggctcaggaccagaggatcagatggtacctgctgagcatggggtctaacgagaatatccactccatccatttcagcggacacgtgtttaccgtccgcaagaaagaagagtacaagatggccctgtacaacctgtatcccggcgtgttcgaaaccgtcgagatgctgccttccaaggctgggatctggcgggtggaatgcctgatcggggagcacctgcatgccggaatgtctaccctgttcctggtgtactccaataagtgtcagacccccctggggatggctagcggacatatccgcgacttccagatcaccgcttccggacagtacggacagtgggctcctaagctggctagactgcactattctggctccatcaacgcttggtctaccaaagagcctttctcctggatcaaggtggacctgctggctccaatgatcatccatggcatcaaaacccagggggccaggcagaagttctcttccctgtacatcagccagtttatcatcatgtattctctggatgggaagaaatggcagacctacagaggcaattccaccgggaccctgatggtgttctttggcaacgtcgacagctctgggatcaagcacaacatcttcaatccccctatcatcgcccgctacatccggctgcacccaacccattattccatccgcagcaccctgcggatggagctgatggggtgcgatctgaacagctgttctatgcccctgggaatggagtctaaggccatctccgacgctcagatcaccgcctccagctacttcaccaatatgtttgctacctggtccccaagcaaggctagactgcatctgcagggaagaagcaacgcttggagaccacaggtgaacaatcccaaggagtggctgcaggtcgacttccagaaaaccatgaaggtgaccggagtcaccacccagggcgtgaaaagcctgctgacctctatgtacgtcaaggagttcctgatctcttccagccaggacgggcaccagtggaccctgttctttcagaacggaaaggtgaaagtcttccagggcaatcaggattcctttacccctgtggtcaacagcctggacccacccctgctgaccaggtacctgagaatccacccacagtcctgggtgcatcagatcgctctgaggatggaagtcctgggctgcgaggcccaggacctgtattga

SEQ ID NO: 14 - DNA, 4374 bp, synthetic construct
  misc_feature 1..4374: note = synthetic lcoHSQ CpGs removed
  source 1..4374: mol_type = other DNA
atgcagattgaactgtctacctgtttctttctgtgcctgctgaggttttgtttttctgctaccagaagatactacctgggagctgtggaactgagctgggattacatgcagtctgacctgggagagctgcctgtggatgctagattcccacctagagtccctaagtccttccccttcaacacctctgtggtctacaagaaaaccctgtttgtggagtttacagaccacctgttcaacattgctaagcctagaccaccatggatgggactgctgggaccaaccatccaggcagaggtgtatgacacagtggtcatcaccctgaaaaacatggcttctcaccctgtgtccctgcatgctgtgggagtctcctactggaaggcctctgaaggggctgagtatgatgatcagaccagccagagggaaaaagaggatgataaggtgttccctggagggtcccatacctatgtgtggcaggtcctgaaggagaatggaccaatggcttctgaccctctgtgcctgacctactcttatctgtcccatgtggacctggtcaaggatctgaactctggcctgattggggctctgctggtgtgtagggaagggtccctggccaaggagaaaacccagaccctgcataagttcatcctgctgtttgctgtgtttgatgaaggaaaaagctggcactctgagaccaagaactctctgatgcaggacagggatgctgcttctgccagagcttggcccaagatgcacacagtgaatggctatgtcaataggagcctgcctggactgattggctgccacagaaagtctgtgtattggcatgtcattggaatgggcaccacccctgaagtgcacagcatcttcctggaggggcatacctttctggtcaggaaccacaggcaggctagcctggagatctctccaatcaccttcctgacagcccagaccctgctgatggacctgggacagttcctgctgttttgccacatctccagccaccagcatgatggcatggaggcttatgtgaaagtggactcctgtcctgaggaacctcagctgaggatgaagaacaatgaggaagctgaagactatgatgatgacctgacagactctgagatggatgtggtcaggtttgatgatgataactctccctcctttatccagatcaggtctgtggccaagaaacaccctaagacctgggtccattacattgctgctgaggaagaggactgggattatgctccactggtgctggcccctgatgatagatcctacaaaagccagtatctgaacaatggaccccagaggattggcagaaagtacaagaaagtgaggttcatggcttatacagatgagacctttaagaccagagaagccatccagcatgagtctgggatcctgggacctctgctgtatggggaagtgggggacaccctgctgatcatcttcaagaaccaggccagcaggccttacaatatctatccacatggcatcacagatgtgagacctctgtactccaggaggctgccaaagggggtgaaacacctgaaggacttcccaatcctgcctggggaaatctttaagtataaatggacagtcacagtggaggatgggcccaccaagtctgaccctaggtgcctgaccagatactattcttcctttgtgaatatggagagagacctggcttctggactgattggacccctgctgatctgttacaaagagtctgtggatcagaggggcaaccagatcatgtctgacaagaggaatgtgatcctgttctctgtctttgatgaaaacaggtcttggtacctgacagagaacatccagaggttcctgcctaatccagctggagtgcagctggaagatcctgagttccaggcctctaacatcatgcattccatcaatggctatgtgtttgactccctgcagctgtctgtgtgcctgcatgaggtggcttactggtatatcctgagcattggagcccagacagatttcctgtctgtgttcttttctggctacacctttaagcataaaatggtgtatgaggacaccctgaccctgttcccattttctggagaaactgtgttcatgagcatggagaatcctgggctgtggatcctgggatgccacaactctgatttcaggaatagagggatgacagccctgctgaaagtgagctcttgtgacaagaacacaggagactactatgaagatagctatgaggacatctctgcttatctgctgtccaaaaacaatgccattgagcccaggagcttctctcagaaccctccagtgctgaagaggcaccagagggagatcaccagaaccaccctgcagtctgatcaggaagagattgactatgatgataccatctctgtggaaatgaagaaagaggactttgatatctatgatgaagatgagaaccagtctcccaggtccttccagaagaaaaccagacattactttattgctgctgtggagaggctgtgggactatggcatgtccagctctcctcatgtgctgagaaatagagctcagtctggatctgtcccacagttcaagaaagtggtcttccaggagtttacagatggaagctttacccagccactgtacaggggagaactgaatgagcacctggggctgctgggaccctatatcagggctgaagtggaggataacatcatggtcaccttcaggaatcaggccagcagaccctactctttttattccagcctgatctcctatgaagaggaccagagacagggagctgaaccaagaaaaaactttgtgaagcctaatgagaccaaaacctacttttggaaggtgcagcaccatatggcccctaccaaagatgagtttgattgcaaggcctgggcttatttttctgatgtggatctggagaaggatgtccactctggcctgattgggccactgctggtgtgtcataccaacaccctgaatccagctcatggaaggcaggtgacagtccaggaatttgccctgttctttaccatctttgatgagaccaagagctggtacttcacagaaaacatggagaggaattgcagagccccatgtaacatccagatggaagaccccaccttcaaggagaactacagatttcatgctatcaatgggtatatcatggataccctgccaggactggtcatggctcaggaccagaggatcagatggtacctgctgagcatggggtctaatgagaatatccactccatccatttctctggacatgtgtttacagtaaggaagaaagaagagtacaagatggccctgtacaacctgtatcctggggtgtttgaaacagtggagatgctgccttccaaggctgggatctggagggtggaatgcctgattggggagcacctgcatgctggaatgtctaccctgttcctggtgtactccaataagtgtcagacccccctggggatggcttctggacatatcagggacttccagatcacagcttctggacagtatggacagtgggctcctaagctggctagactgcactattctggctccatcaatgcttggtctaccaaagagcctttctcctggatcaaggtggacctgctggctccaatgatcatccatggcatcaaaacccagggggccaggcagaagttctcttccctgtacatcagccagtttatcatcatgtattctctggatgggaagaaatggcagacctacagaggcaattccacagggaccctgatggtgttctttggcaatgtggacagctctgggatcaagcacaacatcttcaatccccctatcattgccaggtacatcagactgcacccaacccattattccatcaggagcaccctgagaatggagctgatggggtgtgatctgaacagctgttctatgcccctgggaatggagtctaaggccatctctgatgctcagatcacagcctccagctacttcaccaatatgtttgctacctggtccccaagcaaggctagactgcatctgcagggaagaagcaatgcttggagaccacaggtgaacaatcccaaggagtggctgcaggtggacttccagaaaaccatgaaggtgacaggagtcaccacccagggagtgaaaagcctgctgacctctatgtatgtcaaggagttcctgatctcttccagccaggatgggcaccagtggaccctgttctttcagaatggaaaggtgaaagtcttccagggcaatcaggattcctttacccctgtggtcaacagcctggacccacccctgctgaccaggtacctgagaatccacccacagtcctgggtgcatcagattgctctgaggatggaagtcctgggctgtgaggcccaggacctgtattga

SEQ ID NO: 15 - DNA, 4374 bp, synthetic construct
  misc_feature 1..4374: note = synthetic NoCoHSQ
  source 1..4374: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggaccaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagacgcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatccgcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaacctaggagcttctctcagaatccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 16 - DNA, 4375 bp, synthetic construct
  misc_feature 1..4375: note = synthetic mcoHSQ
  source 1..4375: mol_type = other DNA
atgcagattgagctcagcacctgcttctttctgtgcctgctcaggttctgcttttcagccacaaggagatactatctgggagctgtggaactgtcatgggattacatgcagagtgacctgggagagctccctgtggatgctaggttccccccaagggtcccaaagtctttcccttttaataccagtgtggtctataagaaaacactctttgtggaatttactgatcacctgttcaacattgcaaagccaaggcctccctggatgggactgctgggacctaccatccaggctgaggtgtatgacactgtggtcatcacactgaaaaacatggcatctcaccctgtcagcctgcatgcagtgggagtcagctactggaaggcttcagaaggggcagagtatgatgatcagacaagccagagagaaaaagaggatgataaggtgttcccaggagggagccatacttatgtgtggcaggtcctgaaggagaatggcccaatggccagtgacccactgtgcctcacctactcatatctgagtcatgtggacctggtcaaggatctcaactcaggcctgattggggcactgctggtgtgcagggaaggctcactggccaaggagaaaacccagacactgcataagttcatcctgctctttgctgtgtttgatgaagggaaatcttggcacagtgagaccaagaacagtctgatgcaggacagggatgctgcttctgccagagcttggcccaagatgcacacagtgaatggatatgtcaataggtccctgccaggactcattggctgccacagaaagtcagtgtattggcatgtcattggaatgggcaccacaccagaagtgcacagcatcttcctggaggggcatacctttctggtcaggaaccacaggcaggccagcctggagatcagcccaatcaccttcctgacagcccagactctgctcatggatctggggcagttcctgctcttttgccacatcagctcccaccagcatgatggaatggaggcatatgtgaaagtggactcctgcccagaggaaccacagctgaggatgaagaacaatgaggaagctgaagactatgatgatgacctgacagactcagagatggatgtggtcaggtttgatgatgataacagcccctcctttatccagatcagaagtgtggccaagaaacacccaaagacatgggtccattacattgcagctgaggaagaggactgggattatgcacctctggtgctggccccagatgatagatcctacaaatcacagtatctgaacaatggaccccagaggattggcagaaagtacaagaaagtgaggttcatggcctatactgatgaaacatttaagactagagaagctatccagcatgagtcaggcatcctgggaccactgctctatggagaagtgggggacaccctgctcatcatcttcaagaaccaggcttccaggccatacaatatctatcctcatggcatcacagatgtgagaccactctactcaaggagactgcctaagggagtcaaacacctcaaggacttccctatcctgccaggggaaatctttaagtataaatggactgtgacagtggaggatgggcccactaagagtgacccaaggtgcctgaccagatactattcaagttttgtgaatatggaaagggatctggcatcaggactgattggacctctgctcatctgctacaaagagagtgtggatcagaggggcaaccagatcatgtcagacaagaggaatgtgatcctgttcagtgtctttgatgaaaacaggtcttggtatctgacagagaacatccagagattcctgccaaatcctgcaggggtgcagctggaagatccagagtttcaggcctcaaacatcatgcatagtatcaatggatatgtgtttgacagtctgcagctctctgtgtgcctgcatgaagtggcctactggtatatcctgtccattggagctcagacagatttcctgagtgtgttcttttcaggctacacttttaagcataaaatggtctatgaggacacactgactctcttcccttttagtggggaaacagtgtttatgagcatggagaatccagggctgtggattctgggatgccacaacagtgatttcaggaatagaggcatgactgctctgctcaaagtgtctagctgtgacaagaacacaggggactactatgaagattcttatgaggacatcagtgcttatctgctctccaaaaacaatgcaattgaacccagatcattcagtcagaatccacctgtgctgaagaggcaccagagagagatcactaggactaccctgcagtcagatcaggaagagattgactatgatgataccatctcagtggaaatgaagaaagaggactttgatatctatgatgaagatgagaaccagagtccaaggtctttccagaagaaaaccagacattactttattgctgcagtggagaggctgtgggattatggaatgtcctcaagtccacatgtgctgaggaatagggcacagtctggcagtgtccctcagttcaagaaagtggtcttccaggagtttacagatggcagcttcactcagcctctgtacaggggagaactcaatgagcacctggggctgctgggaccctatatcagagctgaagtggaggataacatcatggtcaccttcaggaatcaggcttcaagaccctacagtttttattctagcctgatcagctatgaagaggaccagaggcagggagctgaacctaggaaaaactttgtgaagccaaatgagaccaaaacatacttttggaaggtccagcaccacatggcaccaaccaaagatgagtttgattgcaaggcatgggcctatttttcagatgtggatctggagaaggatgtccacagtggcctcattgggcctctgctggtgtgccatactaacaccctgaatccagctcatggcaggcaggtgacagtccaggagtttgcactgttctttaccatctttgatgagacaaagtcctggtacttcactgaaaacatggagaggaattgcagagctccttgcaacatccagatggaagaccccaccttcaaggagaactacagatttcatgcaatcaatgggtatatcatggatacactgccaggactggtgatggcccaggaccagaggatcagatggtatctgctcagcatggggtccaatgagaatatccactctatccatttcagtggacatgtgtttacagtcagaaagaaagaagagtataaaatggccctgtacaacctctatccaggagtgtttgaaacagtggagatgctgccaagcaaggctgggatctggagggtggaatgcctcattggggagcacctgcatgcaggaatgtcaaccctgtttctggtctacagtaataagtgccagacacctctgggaatggcaagtggacatatcagggatttccagatcactgctagtggacagtatggacagtgggcaccaaagctggctagactccactattcaggctcaatcaatgcttggtccaccaaagagccattctcatggatcaaggtggacctgctggctcctatgatcatccatggcatcaaaacacagggggcaaggcagaagttctcctcactgtacatctctcagtttatcatcatgtatagcctggatggcaagaaatggcagacctacaggggcaatagcacagggactctgatggtgttctttggcaatgtggacagcagtgggatcaagcacaacatcttcaatcccccaatcattgcaaggtacatcagactgcaccccacccattattcaatcaggagtacactcaggatggaactgatggggtgtgatctcaacagttgctctatgccactgggaatggagtccaaggcaatctcagatgcccagatcactgctagctcctacttcactaatatgtttgctacctggagcccctccaaagcaaggctgcacctccagggaaggagcaatgcatggaggcctcaggtgaacaatcccaaggaatggctgcaggtggatttccagaaaactatgaaggtgactggagtcacaactcagggagtgaaaagtctgctcacttctatgtatgtcaaggagttcctgatctcaagttctcaggatggccaccagtggaccctgttctttcagaatggaaaggtgaaagtcttccagggcaatcaggattcctttacaccagtggtcaactcactggaccctcccctgctcactagatatctgagaatccaccctcagagctgggtgcatcagattgctctcagaatggaagtcctgggctgtgaggcacaggacctgtattgag

SEQ ID NO: 17 - DNA, 2903 bp, synthetic construct
  misc_feature 1..2903: note = synthetic 6x-tRNA
  source 1..2903: mol_type = other DNA
gtcgactacgtagaatccggagccgtctttgtcttccagctccatcttttccaccttttgcttaggcagtcccccgagtcgtgtcaaggctgaggagtagaaatggaacagcactaatattaatggcaaaaccgttgtgaaatagggttactttctgtttaagcaaggaaaataaagtaaagcaatgggaaaaaaattaaaagcaaaaggaatggaggtgccggggattgaacccggggcctcgtgcatgctaagcacgcgctctaccactgagctacacccccgtactgaaacggttctctcgagagtatattcaagatcagaatctgacccttttgctaggtttcagaaccattagttgtaatcagccaaggtctattttatttagttatttctgatatctcaaatttaggttttgcgtccctctttgctgacagctgagcaaaccgcattctacaccgaaggccctctattgatggccctgatttaaatcccttccgccgcctgccgcaggtggctagggtctgagcacacttgaacctcacacccgccccaggggtagctccttggttcccttcagccccgatgtgtccctcgtgctttgaaatggaattacagttttggttaaaaacatgccttttccgagttaggaagaatctaaatcgactgaacgccagtctaaaatttcggcgttcccacaccgggagtcgaacccgggccgcctgggtgaaaaccaggaatcctaaccgctagaccatgtgggagacggcaatagcgactccaagcctagacaaattgagtcttctcggtcggcttccgcccactccatcgcgttcatccgtaggcgtcaaacctgctcctgcgcctgcgcggagtctgcagcggtttaaaccgttcaggttcgcattctactgtttctctccttgcaggggcccttgaatctttcttcaatctactctcgcgtgcccgggcgactgggcataaccctacaggttcatgtggggtgggtggcgcgcgctagcggtgaaggtcactcacaattgcgcgctgggcagacgacggcagccattacttttacctcgatcgctgttttcctggatccgcacgggtccaacccgactcatcccaaccaacctgaggtatgaaaaccaggaaagagagctagcaccggagcgttggtggtatagtggtaagcatagctgccttccaagcagttgacccgggttcgattcccggccaacgcaagtcgttttgggtgttttttcccccccccgccttttccttttcgtgttttctgggccccagcatcgttgagggttttcgtgaggttttcctgaggaaacttccgctccgaaaggacccactttccgctacacccgcgaccacggctggaccaccgcgctcctgacggatgcgccctgcaagcccctccaggcgagagcaggccggcctgtgctcagttttgtagcatcaaaactaggatttctcttgttacccccagtcactccattcagttttcgtgtctttcccagctgcatccatcctttcctcattttcgtatgcagccgactttttgtgacatctttgtattcattctctgcaattcagctgacctggccaaggaaacaagatcctaagcgtctttccggcggcgccgtggcttagttggttaaagcgcctgtctagtaaacaggagatcctgggttcgaatcccagcggtgcctccgtgtttcccccacgcttttgccaacattaaacattgtgaggacagttgcagaaactcataacttccatcctacatggtttactcacgtacccatctatcctctcccggtgcatctgccacacgctgttgggtttttgctcttcgtgcacatggtacttgcgcctcgacctgcagttacaccagtcgcatcatctgtacagcgctaaaacctagctgggcgtggtggtcctgcagtcccagctactcgggaggctgaggcaggagaatggcttgaatccaggaggcggaggtggcagtgagccgagattgcgccactgcactccagcctggtgacagagcgagactccatctcaaaaaaaaaaaaaaaaaaaaagtcagaattaggtactaaaagccatatgacatgcctgaacagggacttgaaccctggaccctcagattaaaagtctgatgctctaccaactgagctatccaggcttcttccctgctagtttattttaatgcagtaataaataacagcactttgttaaaaataataaaggtataatctgtgacacatccaaagtggacaagatgaagagataataggtatacaccaggattcccctgggcaaactgggacctcttggtaccctatatattaagagtctcgggttttgttttcacttaagcaaatggttaacgaattagcaggttaagaaaaactgtttccccgtaagaagcagggttctttggtgttcaatgtggagctccgccactcccagcccgggtgaaggaaaactgggaaacagaatgaatgtgattatctattcgaagataaatttccacaaagcatgccgtttgatagtagcttataatgtggaagtaaggcatcctgtcatccggccggttagctcagttggttagagcgtggtgctaataacgccaaggtcgcgggttcgatccccgtactggccaagtattctctgtggcttttatcaccagaatggatagtaacccagacatcgatctaaacgtgtacctgtgtgtttctccaggcttaactttgccccgagaaaacggatctgtgaatttggtgcgccctcgcttactcgacagcggttaatttgaacggggacgtttctttccgctgcctccaaggcatacccacatcctaccacgatggtggcggccgcaa

SEQ ID NO: 18 - protein, 1467 aa, synthetic construct
  REGION 1..1467: note = synthetic HP44/OL aa
MQLELSTCVFLCLLPLGFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYISAEEEDWDYAPAVPSPSDRSYKSLYLNSGPQRIGRKYKKARFVAYTDVTFKTRKAIPYESGILGPLLYGEVGDTLLIIFKNKASRPYNIYPHGITDVSALHPGRLLKGWKHLKDMPILPGETFKYKWTVTVEDGPTKSDPRCLTRYYSSSINLEKDLASGLIGPLLICYKESVDQRGNQMMSDKRNVILFSVFDENQSWYLAENIQRFLPNPDGLQPQDPEFQASNIMHSINGYVFDSLQLSVCLHEVAYWYILSVGAQTDFLSVFFSGYTFKHKMVYEDTLTLFPFSGETVFMSMENPGLWVLGCHNSDLRNRGMTALLKVYSCDRDIGDYYDNTYEDIPGFLLSGKNVIEPRSFAQNSRPPSASAPKPPVLRRHQRDISLPTFQPEEDKMDYDDIFSTETKGEDFDIYGEDENQDPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRHNFVQPNETRTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRIECLIGEHLQAGMSTTFLVYSKKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 19 - DNA, 4401 bp, synthetic construct
  misc_feature 1..4401: note = synthetic HP44/OL NT
  source 1..4401: mol_type = other DNA
atgcagctagagctctccacctgtgtctttctgtgtctcttgccactcggctttagtgccatcaggagatactacctgggcgcagtggaactgtcctgggactaccggcaaagtgaactcctccgtgagctgcacgtggacaccagatttcctgctacagcgccaggagctcttccgttgggcccgtcagtcctgtacaaaaagactgtgttcgtagagttcacggatcaacttttcagcgttgccaggcccaggccaccatggatgggtctgctgggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtcttacctactcatacctgtctcacgtggacctggtgaaagacctgaattcgggcctcattggagccctgctggtttgtagagaagggagtctgaccagagaaaggacccagaacctgcacgaatttgtactactttttgctgtctttgatgaagggaaaagttggcactcagcaagaaatgactcctggacacgggccatggatcccgcacctgccagggcccagcctgcaatgcacacagtcaatggctatgtcaacaggtctctgccaggtctgatcggatgtcataagaaatcagtctactggcacgtgattggaatgggcaccagcccggaagtgcactccatttttcttgaaggccacacgtttctcgtgaggcaccatcgccaggcttccttggagatctcgccactaactttcctcactgctcagacattcctgatggaccttggccagttcctactgttttgtcatatctcttcccaccaccatggtggcatggaggctcacgtcagagtagaaagctgcgccgaggagccccagctgcggaggaaagctgatgaagaggaagattatgatgacaatttgtacgactcggacatggacgtggtccggctcgatggtgacgacgtgtctccctttatccaaatccgctcggttgccaagaagcatcccaaaacctgggtgcactacatctctgcagaggaggaggactgggactacgcccccgcggtccccagccccagtgacagaagttataaaagtctctacttgaacagtggtcctcagcgaattggtaggaaatacaaaaaagctcgattcgtcgcttacacggatgtaacatttaagactcgtaaagctattccgtatgaatcaggaatcctgggacctttactttatggagaagttggagacacacttttgattatatttaagaataaagcgagccgaccatataacatctaccctcatggaatcactgatgtcagcgctttgcacccagggagacttctaaaaggttggaaacatttgaaagacatgccaattctgccaggagagactttcaagtataaatggacagtgactgtggaagatgggccaaccaagtccgatcctcggtgcctgacccgctactactcgagctccattaatctagagaaagatctggcttcgggactcattggccctctcctcatctgctacaaagaatctgtagaccaaagaggaaaccagatgatgtcagacaagagaaacgtcatcctgttttctgtattcgatgagaatcaaagctggtacctcgcagagaatattcagcgcttcctccccaatccggatggattacagccccaggatccagagttccaagcttctaacatcatgcacagcatcaatggctatgtttttgatagcttgcagctgtcggtttgtttgcacgaggtggcatactggtacattctaagtgttggagcacagacggacttcctctccgtcttcttctctggctacaccttcaaacacaaaatggtctatgaagacacactcaccctgttccccttctcaggagaaacggtcttcatgtcaatggaaaacccaggtctctgggtccttgggtgccacaactcagacttgcggaacagagggatgacagccttactgaaggtgtatagttgtgacagggacattggtgattattatgacaacacttatgaagatattccaggcttcttgctgagtggaaagaatgtcattgaacctaggagctttgcccagaattcaagaccccctagtgcgagcgctccaaagcctccggtcctgcgacggcatcagagggacataagccttcctacttttcagccggaggaagacaaaatggactatgatgatatcttctcaactgaaacgaagggagaagattttgacatttacggtgaggatgaaaatcaggaccctcgcagctttcagaagagaacccgacactatttcattgctgcggtggagcagctctgggattacgggatgagcgaatccccccgggcgctaagaaacagggctcagaacggagaggtgcctcggttcaagaaggtggtcttccgggaatttgctgacggctccttcacgcagccgtcgtaccgcggggaactcaacaaacacttggggctcttgggaccctacatcagagcggaagttgaagacaacatcatggtaactttcaaaaaccaggcgtctcgtccctattccttctactcgagccttatttcttatccggatgatcaggagcaaggggcagaacctcgacacaacttcgtccagccaaatgaaaccagaacttacttttggaaagtgcagcatcacatggcacccacagaagacgagtttgactgcaaagcctgggcctacttttctgatgttgacctggaaaaagatgtgcactcaggcttgatcggcccccttctgatctgccgcgccaacaccctgaacgctgctcacggtagacaagtgaccgtgcaagaatttgctctgtttttcactatttttgatgagacaaagagctggtacttcactgaaaatgtggaaaggaactgccgggccccctgccatctgcagatggaggaccccactctgaaagaaaactatcgcttccatgcaatcaatggctatgtgatggatacactccctggcttagtaatggctcagaatcaaaggatccgatggtatctgctcagcatgggcagcaatgaaaatatccattcgattcattttagcggacacgtgttcagtgtacggaaaaaggaggagtataaaatggccgtgtacaatctctatccgggtgtctttgagacagtggaaatgctaccgtccaaagttggaatttggcgaatagaatgcctgattggcgagcacctgcaagctgggatgagcacgactttcctggtgtacagcaagaagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 20 - protein, 1457 aa, synthetic construct
  REGION 1..1457: note = synthetic HP46/SQ aa
MQLELSTCVFLCLLPLGFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 21 - DNA, 4368 bp, synthetic construct
  misc_feature 1..4368: note = synthetic HP46/SQ NT
  source 1..4368: mol_type = other DNA
atgcagctagagctctccacctgtgtctttctgtgtctcttgccactcggctttagtgccatcaggagatactacctgggcgcagtggaactgtcctgggactaccggcaaagtgaactcctccgtgagctgcacgtggacaccagatttcctgctacagcgccaggagctcttccgttgggcccgtcagtcctgtacaaaaagactgtgttcgtagagttcacggatcaacttttcagcgttgccaggcccaggccaccatggatgggtctgctgggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtcttacctactcatacctgtctcacgtggacctggtgaaagacctgaattcgggcctcattggagccctgctggtttgtagagaagggagtctgaccagagaaaggacccagaacctgcacgaatttgtactactttttgctgtctttgatgaagggaaaagttggcactcagcaagaaatgactcctggacacgggccatggatcccgcacctgccagggcccagcctgcaatgcacacagtcaatggctatgtcaacaggtctctgccaggtctgatcggatgtcataagaaatcagtctactggcacgtgattggaatgggcaccagcccggaagtgcactccatttttcttgaaggccacacgtttctcgtgaggcaccatcgccaggcttccttggagatctcgccactaactttcctcactgctcagacattcctgatggaccttggccagttcctactgttttgtcatatctcttcccaccaccatggtggcatggaggctcacgtcagagtagaaagctgcgccgaggagccccagctgcggaggaaagctgatgaagaggaagattatgatgacaatttgtacgactcggacatggacgtggtccggctcgatggtgacgacgtgtctccctttatccaaatccgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagacgcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatccgcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaacctaggagcttctctcagaatccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctc

SEQ ID NO: 22 - protein, 1467 aa, synthetic construct
  REGION 1..1467: note = synthetic HP47/OL aa
MQLELSTCVFLCLLPLGFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFAQNSRPPSASAPKPPVLRRHQRDISLPTFQPEEDKMDYDDIFSTETKGEDFDIYGEDENQDPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRHNFVQPNETRTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRIECLIGEHLQAGMSTTFLVYSKKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 23 - DNA, 4401 bp, synthetic construct
  misc_feature 1..4401: note = synthetic HP47/OL NT
  source 1..4401: mol_type = other DNA
atgcagctagagctctccacctgtgtctttctgtgtctcttgccactcggctttagtgccatcaggagatactacctgggcgcagtggaactgtcctgggactaccggcaaagtgaactcctccgtgagctgcacgtggacaccagatttcctgctacagcgccaggagctcttccgttgggcccgtcagtcctgtacaaaaagactgtgttcgtagagttcacggatcaacttttcagcgttgccaggcccaggccaccatggatgggtctgctgggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtcttacctactcatacctgtctcacgtggacctggtgaaagacctgaattcgggcctcattggagccctgctggtttgtagagaagggagtctgaccagagaaaggacccagaacctgcacgaatttgtactactttttgctgtctttgatgaagggaaaagttggcactcagcaagaaatgactcctggacacgggccatggatcccgcacctgccagggcccagcctgcaatgcacacagtcaatggctatgtcaacaggtctctgccaggtctgatcggatgtcataagaaatcagtctactggcacgtgattggaatgggcaccagcccggaagtgcactccatttttcttgaaggccacacgtttctcgtgaggcaccatcgccaggcttccttggagatctcgccactaactttcctcactgctcagacattcctgatggaccttggccagttcctactgttttgtcatatctcttcccaccaccatggtggcatggaggctcacgtcagagtagaaagctgcgccgaggagccccagctgcggaggaaagctgatgaagaggaagattatgatgacaatttgtacgactcggacatggacgtggtccggctcgatggtgacgacgtgtctccctttatccaaatccgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagacgcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatccgcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaacctaggagctttgcccagaattcaagaccccctagtgcgagcgctccaaagcctccggtcctgcgacggcatcagagggacataagccttcctacttttcagccggaggaagacaaaatggactatgatgatatcttctcaactgaaacgaagggagaagattttgacatttacggtgaggatgaaaatcaggaccctcgcagctttcagaagagaacccgacactatttcattgctgcggtggagcagctctgggattacgggatgagcgaatccccccgggcgctaagaaacagggctcagaacggagaggtgcctcggttcaagaaggtggtcttccgggaatttgctgacggctccttcacgcagccgtcgtaccgcggggaactcaacaaacacttggggctcttgggaccctacatcagagcggaagttgaagacaacatcatggtaactttcaaaaaccaggcgtctcgtccctattccttctactcgagccttatttcttatccggatgatcaggagcaaggggcagaacctcgacacaacttcgtccagccaaatgaaaccagaacttacttttggaaagtgcagcatcacatggcacccacagaagacgagtttgactgcaaagcctgggcctacttttctgatgttgacctggaaaaagatgtgcactcaggcttgatcggcccccttctgatctgccgcgccaacaccctgaacgctgctcacggtagacaagtgaccgtgcaagaatttgctctgtttttcactatttttgatgagacaaagagctggtacttcactgaaaatgtggaaaggaactgccgggccccctgccatctgcagatggaggaccccactctgaaagaaaactatcgcttccatgcaatcaatggctatgtgatggatacactccctggcttagtaatggctcagaatcaaaggatccgatggtatctgctcagcatgggcagcaatgaaaatatccattcgattcattttagcggacacgtgttcagtgtacggaaaaaggaggagtataaaatggccgtgtacaatctctatccgggtgtctttgagacagtggaaatgctaccgtccaaagttggaatttggcgaatagaatgcctgattggcgagcacctgcaagctgggatgagcacgactttcctggtgtacagcaagaagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 24 - protein, 1457 aa, Homo sapiens
MQIELSTCFFLCLLRFCFSATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 25 - DNA, 4371 bp, Homo sapiens
  source 1..4371: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagacgcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatccgcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaacctaggagcttctctcagaatccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 26 - protein, 1467 aa, synthetic construct
  REGION 1..1467: note = synthetic HP63/OL aa
MQLELSTCVFLCLLPLGFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRSQNPPVLKRHQREITRTTLQSDQEEIDYDDTISVEMKKEDFDIYDEDENQSPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRKNFVKPNETKTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRNRCLIGEHLQAGMSTTFLVYSKKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 27 - DNA, 4398 bp, synthetic construct
  misc_feature 1..4398: note = synthetic HP63/OL NT
  source 1..4398: mol_type = other DNA
atgcagctagagctctccacctgtgtctttctgtgtctcttgccactcggctttagtgccatcaggagatactacctgggcgcagtggaactgtcctgggactaccggcaaagtgaactcctccgtgagctgcacgtggacaccagatttcctgctacagcgccaggagctcttccgttgggcccgtcagtcctgtacaaaaagactgtgttcgtagagttcacggatcaacttttcagcgttgccaggcccaggccaccatggatgggtctgctgggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtcttacctactcatacctgtctcacgtggacctggtgaaagacctgaattcgggcctcattggagccctgctggtttgtagagaagggagtctgaccagagaaaggacccagaacctgcacgaatttgtactactttttgctgtctttgatgaagggaaaagttggcactcagcaagaaatgactcctggacacgggccatggatcccgcacctgccagggcccagcctgcaatgcacacagtcaatggctatgtcaacaggtctctgccaggtctgatcggatgtcataagaaatcagtctactggcacgtgattggaatgggcaccagcccggaagtgcactccatttttcttgaaggccacacgtttctcgtgaggcaccatcgccaggcttccttggagatctcgccactaactttcctcactgctcagacattcctgatggaccttggccagttcctactgttttgtcatatctcttcccaccaccatggtggcatggaggctcacgtcagagtagaaagctgcgccgaggagccccagctgcggaggaaagctgatgaagaggaagattatgatgacaatttgtacgactcggacatggacgtggtccggctcgatggtgacgacgtgtctccctttatccaaatccgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaacctaggagcttctcccagaattcaagacaccctagcactaggtctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagagaacccgacactatttcattgctgcggtggagcagctctgggattacgggatgagcgaatccccccgggcgctaagaaacagggctcagaacggagaggtgcctcggttcaagaaggtggtcttccgggaatttgctgacggctccttcacgcagccgtcgtaccgcggggaactcaacaaacacttggggctcttgggaccctacatcagagcggaagttgaagacaacatcatggtaactttcaaaaaccaggcgtctcgtccctattccttctactcgagccttatttcttatccggatgatcaggagcaaggggcagaacctcgaaaaaactttgtcaagcctaatgaaaccaaaacttacttttggaaagtgcagcatcacatggcacccacagaagacgagtttgactgcaaagcctgggcctacttttctgatgttgacctggaaaaagatgtgcactcaggcttgatcggcccccttctgatctgccgcgccaacaccctgaacgctgctcacggtagacaagtgaccgtgcaagaatttgctctgtttttcactatttttgatgagacaaagagctggtacttcactgaaaatgtggaaaggaactgccgggccccctgccatctgcagatggaggaccccactctgaaagaaaactatcgcttccatgcaatcaatggctatgtgatggatacactccctggcttagtaatggctcagaatcaaaggatccgatggtatctgctcagcatgggcagcaatgaaaatatccattcgattcattttagcggacacgtgttcagtgtacggaaaaaggaggagtataaaatggccgtgtacaatctctatccgggtgtctttgagacagtggaaatgctaccgtccaaagttggaatttggcggaatagatgcctgattggcgagcacctgcaagctgggatgagcacgactttcctggtgtacagcaagaagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 28 - DNA, 5508 bp, Homo sapiens
  source 1..5508: mol_type = other DNA
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcaccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgccccccgacatggcacaagtcactgtgggcccggggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgactgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagaggtgctgctccggagaggggctgcagatccccaccctctcccctgcacctcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacggacggagcccatgcaggtggccctgcactgcaccaatggctctgttgtgtaccatgaggttctcaatgccatggagtgcaaatgctcccccaggaagtgcagcaagtga

SEQ ID NO: 29 - DNA, 6648 bp, Ovis aries
  source 1..6648: mol_type = other DNA
atgtttcccaccaggctcgcgaggctgctgcttgctgtggccctcactttgccaggggccctttgtggagaaggtgctcctggcaagtcatcgatggcccggtgcagcctcttcggagctgacttcatcaacacctttgatgagagcatgtacagctttgcgggagactgtagttacctcctggcaggggattgcaagacacactccttttcaatcgtaggggacttccaagctggtagaagagtgggtctctctgtgtaccttggggaatttttcgacatccatgtgtttgtcaacggtactgtgctgcaggggggccagcatgtctccatgccctatgccaccagagggctgtacctggacaccgaggctgggtaccacaagctgtccagcgagtcttatggctttgtggccaggatcgacagcagcgggaacttccaaatcctgctgtcggacagacacttcaacaagacctgtgggctgtgcggtgactttaacatcttcgccgaagatgacttcaggactcaagaaggaaccctgacctcagacccctacgactttgcaaactcctgggccctgagcagtgaggagcagcggtgtccacgggtgtcccctcccagcagctcctgcaacatctcctctgagctgcagaagggcctgtgggagcagtgccagcttctgaagacggcctccgtgttcgcccgctgccacgccctggtggaccccgagcctttcgtggccctgtgtgagcggatgctgtgcgcatgcgcccaggggctgcgctgcccctgcccggtgctcctggagtacgcccgcgcctgcgcccagcaagggatgctgctgtacggctgggcggaccacagctcctgccgaccggactgccccgcgggcatggagtacaaggagtgtgtgtccccatgccacaggacctgccggagcctgagtatcaccgaagtgtgtcgggagcagtgtgtggatggctgcagctgccctgagggacagctcctggatgaaggccgctgtgtggaaagtgccgagtgtccctgtgtgcatgctggaaagccataccctcctggcgcctccctctcgcgagactgcaacacctgcatctgccgaaacagccagtgggtctgcagcaatgaggactgtccaggagagtgtctcatcacaggacaatcccacttcaagagctttgacgacaggcacttcaccttcagcggggtctgccagtacctgctggcccaggactgccaggaccactccttctccgtcatcatagagactgttcagtgtgctgacgaccctgatgcggtctgcacccgctccgtcaccgtccgcctgcccagcccgcaccacggcctcctgaagctgaagcacgggggtggagtcgccctggatggccaggacgtccagattcccctcctgcaaggtgacctccggatccagcacactgtgacggcctccctgcacctcatcttcggggaggacctgcagatagactgggacggtcgcgggaggctgctgctgaagctgtccccggtctacgcggggaggacctgcgggctgtgcgggaattacaacggcaaccagagggatgacttcctgacgcccgcgggcctggtcgagcccctggtggagcactttggaaactcctggaagctacgtgcagactgtgaggacttgcaggagcagcccagtgacccctgcagcctcaacccgcgcctgaccaagttcgcagaccaggcctgcgccatcctgacgtcccgcaagttcgaggcctgccacagcgccgtgggcccgctgccctacctgcgcaactgccgcttcgacgtgtgcgcctgctccgatggcagagactgcctgtgcgacgcggtggccaactacgcggcggcctgcgccaggaggggcgtgcacatcgggtggcgggagcccagcttctgtgcactgagctgcccacacggccaggtgtaccagcagtgtgggaccccctgcaacctcacctgccgctcactctcccacccggacgaggaatgcactgaggtctgtctggagggctgcttctgccctgctgggctcttcctggatgagacggggtcctgtgtgcccaaggcccagtgcccctgttactatgacggcgagatcttccaacctgaagacatcttctcggaccatcacaccatgtgctactgcgaagatggcttcatgcactgctccacgagcggagccccggggagcctgctgcctgaagcagtcctcagcagccctctgtcccaccgcagcaaaaggagcctgtcctgccggccccccatggtcaagctggtgtgccctgctgacaacccgagggccgaagggctcgaatgcaccaagacctgccagaactatgacctggaatgcgtgagcacgggctgtgtgtccggctgcctctgccccccgggcatggtccggcatgagaacaggtgtgtggccctggaaaggtgcccctgcttccaccagggcagagagtacgcccccggagacagggtgaaggccgactgcaacacctgcgtctgtcaggaccggaagtggaactgtacggaccgcgtgtgtgatgctggctgctctgccgtgggcctggctcactacttcacctttgatgggctcaagtacctgttcccgggggagtgccagtacgtcctggtacaggaccactgcggtagtaaccctgggaccttccgggtcctggtggggaatgaggggtgcagcgtcccctccctgaagtgcaggaagcgcatcaccatcctggtggagggaggagagatcgagctgtttgacggggaggtgaacgtgaagaagcccatgaaggatgagacgcacttcgaggtggtggaatctggccggtacatcactgtgctgctgggcaaggccctctctgtggtctgggacgggcacctggccatctctgtgttcctgaagcggatgtaccaggagcgggtatgcggcctgtgtgggaatttcgatggcgtccagaacaatgacctcaccagtagcagcctccaagtggaggaagaccctgtggactttgggaattcctggaaagtgagcccgcattgcgctgacacccagaaagtgccgctggactcggcccctgccacctgccacaagaacgtcatgaagcagaccatggtggattcctcctgcagggtcctcaccagtgatgttttccgggagtgcaacaggctggtgaaccccgagccgtacctggatgtttgcatctacgacagctgctcctgcgagtccatcggggactgcgcctgcttctgtgacaccatcgccgcctacgcccacgagtgtgcccagcacggcgaagtggtgacctggaggacagccacactgtgcccccagaattgtgaggagcggaacctgaaggagagtgggtaccaatgtgagtggcgctacaacagctgtgctcccgcctgtccagtcacgtgccagcacccagagcccctggcctgccccgtgcagtgcgtggagggctgccacgcacactgcccgcctgggaaaatcctggacgagcttttgcagacctgtgtcaaccccgaggactgccctgtgtgccaggtggagggccggcgcttagcctccgggaagaaagtgaccctgaaccctggggaccctgagcattgccagctctgtcactgtgatggtgtcagcctcacttgtgaagcctgcagggagccaggaggcctgcccgtgccccccaccgaaggcccggtcagccccacaaccccgtacgtggaggacaccccggagccacccctgcacgacttcttctgcagcaaactgctggacctggtcttcctgctggacggctcctccaagctgtctgaggccgacttcgagacgctgaaggcgttcgtggtgggcatgatggagcgtctgcacatttcccagaaacgcatccgtgtggccgtggtggagtaccacgatggctcccatgcctaccttgcactgcaagaccggaagcggccatccgagctgcggcgcattgccgggcaggtgaagtacgcgggcagcgaggtggcttccaccagcgaggtcttgaagtacacgctcttccagatcttcggcaggattgaccggcccgaggcctctcgcgtggccctgctgcttacggccagccaggagccccccaggctggcccggaacttggtccgctacctccagggcctgaagaagaagaaggtctccgtggtcccggtgggcatcgggccccacgtcagcctcaagcagatccgcctcatcgagaagcaggcgtctgagaacaaagcctttgtgctaagcggtgtgcacgagctggagcagcggatgaacgagattgtcggctacctctgtgacctcgcccccgaggtgcctgccccgaccccgacgcgacatcctctcattgcgcaggtcactgtggcgccgcagctcctgggtccttcgccaccaggacccaagaggagctctgtggtcctggatgtggcattcctcctggaaggctcggatgaggtaggcgaggccaacttcaacaggagcgcagagtttgtggaggaggtgatccgacgcatggacgttggccgggacggcatccatgtcacggtgctgcagtactcgtacacggtgaccgtggagcactcgttcagggagccacagtccaaggaggtggtcctgcagcgactccatgaagtccgctaccggggtggcaaccagacgaacacggggctggccctgcagtacctgtcggagcacagcttctccgccagccagggggaccgggagcaggcgcccaacctggtctacatggtgacgggcagcccggcctcggacaagatccagcggatgccaggagacatccagctcgtgcccatcggcgtgggcccccgtgtggacgtgcaggagctggagagggtcagctggccccagacccccatcttcatccaggacttcgagaggctcccccgagaagctccggatctggtgctgcagcggtgctgctccgaagacggcccgcacctccccaccctcgcccctgccccagactgcagccagcccctggatgtagtcctcctcctggatggctcctccacccctccagcctcttactttgacgaaatgaagagtttcgccaaggctttcatctcaaaagccaacctaggccctcagcttacccaggtgtcagtcctgcagtacgggagcatcaccaacgtcgacgtatcctggaatgtgcacgtggacaaagcccacctgctgagccttgtggaccccatgcagcgcgagggaggccccagccgagtcggggaggcgttgtccttcgcggtgcgctacatcacgtcccaagtccacggtgccaggcccggggcctccaaggtggtggtgatcctggtcacaggctcctccatggactcagtggaggcggccgccgctgctgccagatccaaccgagtggctgtgttccccatcgggatcggggaccagtatgacgcagcccagctgagggtcttggcgggcccgggggccagctccaatgtggcagagctccagcggattgaagacctccccagcatggttgcccttggcaactccttcttccagaggctatgctctgggttcgtcagtgtttgcgtggatgaggacgggaatgagaggaggcctggggacgtctggaccttgctggatcagtgccacacagtgacttgcctgccagatggccagaccttgctgaagagtcaccgggtcaactgtgatcaggggccacagccatcatgccccgacggccagatcccgctcaggatggaggaagcctgtggctgccgctgggcctgtccctgtgtgtgcacaggcagctccactcggcacatcgtgacctttgatggacggaatttcaagctgaccggcaactgctcatacgttctgtttcacaacaaggagcaggacttggaggtgattctccataacggattctgcagcgctggggcgaggcaggcctgcatgaaatctgtggaggtgaagcacagcggcctctcggttgagctccgcagcaacatggaggtgatggtgaatgggagactggtctctgtcccttacctgggtggggacatggaggtcagagtctatggtaccatcatgttcgaggtcagattcaaccttctgggccacatcctctccttcaccccacgtgatgagacactccaggatggctgtgacagtcacttctgcaaagtcaacaagagaggagagttcatttgggagaagagggtcatgggctgcccgcccttcaacgaacacaaatgtctggctgagggggggaaagtcatgaaaattcctggcacgtgctgtgacacatgtgaggagcccgagtgcaaggacatcacagccagggtgcagtacatcaaggtgggagactgcaaatctgaagaggaagtggacattaactactgccagggaagatgcaccagcaaagccctgtactccatcgacacggaggacgtgcaagaccagtgttcctgctgctcgcccacgcgcacggagcccatgtcagtgcccctgcgctgcaccaacggctccatcattcaccatgtgatcctcaacgccctgcagtgcaagtgctcatccaggaagtgccgcccgtga

SEQ ID NO: 30 - protein, 24 aa, synthetic construct
  REGION 1..24: note = synthetic porcine-derived linker sequence
SFAQNSRPPSASAPKPPVLRRHQR

SEQ ID NO: 31 - protein, 14 aa, synthetic construct
  REGION 1..14: note = synthetic human linker sequence
SFSQNPPVLKRHQR

SEQ ID NO: 32 - protein, 6 aa, synthetic construct
  REGION 1..6: note = synthetic 6-His tag
HHHHHH

SEQ ID NO: 33 - DNA, 4350 bp, synthetic construct
  misc_feature 1..4350: note = Synthetic truncated ovine (sheep) gene
  source 1..4350: mol_type = other DNA
atgcacatcaagctctgtacctgcctctttctgtgcctctggccatgcagcttcagtgccatcagaagatactacctgggtgcagtggaactgtcctgggactatacgcgaagtgaactgctcagtgagctgcatgtgaacacgaggtttcctcccagagtgcccaaaccttttccattcaacacatcagtcatgtacagaaagactgtgtttgtagagttcacggatcaactttttaacatcgccaagcccaggccaccatggatgggtctgctgggtccagctatccaggctgaggtttatgacaccgtggtcattacatttaagaatatggcttctcatcctgttagtcttcatgctattggcgtatcctactggaaatcttctgaaggtgctgcatataaggatgaaaccagccaaagggagaaggaagatgacaaagtcattcctggtaaaagtcatacctacgtctggcatatcctgaaagaaaacggtccaacagcctctgacccaccatgtctcacctactcatatctttctcatgtggacctggtgaaagacgtgaactcaggtctcatcggagccctgctaatttgtagggaagggactctgatcaaagaaaggacacagaccttgcacaaattcgtactactgtttgctgtatttgatgaagggaaaagttggcactcgggaaaaaatgagtccttgacacatgttatggattctgcctctgtactgcacaccatcaatggctatataaacaggtctctgccaggtctgattggatgtcataagaaatcagtctattggcatgtgattggaatgggcaccaccccagaagtgcactcaattttcctcgaaggccacacatttctcgtgaggaaccatcgccaggcttccttggagatctcaccaataactttccttaccgctcagacagtcctgatggactgtggccagtttctactgttttgtcgtatctcttcccaccaacatgatggtatggaagcttatgtcaaagtagatagttgcccagaggaaccccgactatggatgaaaaataaccaagaagaagattacgatgatggtttggatgactctgacatggatgtggtcaggttcgatggtgacagtgtgcctccctttatccaaatgcgctcagttgcaaagaagcatcctaaaacctgggtccactacattgctgctgaagaggatgactgggactatgccccctcggtcctcacctccaatgacagaagttataaaagtctgtatctgaaccacggtcctcagcggattggtaggaagtacagaaaagtacgatttatagcttacacagatggaacatttaagactcgtgaagctattcagcatgaatcagggatcctggggcctttactttatggagaagttggagacacacttttgattatatttaagaatcaagcaagccggccatataacatctaccctcatggaatcactgatgtcagtcctttgcactcagggagatttccaaaaggtgtgaaacatttgaaagacatgccaattctgccaggagaagtgttcaagtataaatggacagtgactgtagaagatgggccaactaaatcagatcctcggtgtctgacccgatattactcgagtttcattaacttagagaaagatctagcttcaggactcattggccccctcctcatctgttacaaagaatctgtagatcaaagaggaaaccagatgatgtcagacaagagaaatgtcatcctgttttctgtatttgatgaaaacaaaagttggtacctcacagagaatattcaacgcttcctccccagtggagtacagccccaggatccagagttccaagtctccaatgtcatgcacagcatcaatggctatgtttttgatagcttgcagctgtcggtttgtttgcatgaggtggcgtactggtacattctaagtgttggagcccaaattgacttcctctctgtcttcttctctggatataccttcaaacacaaaatggtctatgaagacacactcaccctattccccttctcgggagaaactgtcttcatgtcaatggaaaatccaggtctgtgggttctggggtgccacaactcagactttcgaaacagaggcatgacagccttactgaaggttgatagttgtgacaggaacgttggcgattattatgacacatatgaagctattccaaccttcctgctgagtgaaaacaatgtcattgaacccagaagcttctcccagaatccaccaagcttgaaacgccatcaaagggagataacccttactacttttcagccagagccagacaaaactgactatgatgatactttgtcgattgaaacaaagagagaagattttgacatttatggtgaagatgaaaatcaggacccccgcagctttcaaaagagaacacgccactattttattgctgcagtggagcggctctgggattatgggatgagtagatccccccacgcactaagaaacaggtctcagaatggaggagtccctcagttcaaaaaggtggtgttcgaggaatttactgatggctcctttactcaggccgtataccgtggacaattaaatgaacacctgggactcttgggaccatatataagagcagaagtggaagacaatatcatggtaactttcaaaaaccaggcctctcgtccctactccttctattctagccttatttcttataacggagatcagagacaaggagcagaacctcgaaaaaagtttgtcaagcctaatgaaacccaaagctacttttggaaagtgcagcaccatatggcacccaccaaagatgagtttgactgcaaagcctgggcttacttttctgatgttgatctggaaaaagatgtgcactcaggcttgattggccccattctgatctgccgtgtggacacgctgagtgctgctcatgggagacaagtgacagtacaggaattcgctctgtttttcaccatttttgatgagaccaagagctggtactttgccgaaaacatggcaaggaactgcgtggcaccctgccatgtccagccagaggaccctactttccaagaaaagtatcgcttccatgcaatcaatggctacgtgatggatacactccctggcttagtcatggctcagcatcaaaggattaggtggtatctgctcagcatgggcagcaatgaaaatatccattccattcatttcagtggccatgtgttcactgtgagaaaaacggaggagtataaaatggcggtctacaatctctacccaggtgtctttgagaccgtggaaatgctaccatccaaggttgggacttggcggatagaatgtcttattggcgagcacctacaagctgggatgagcactctcttcctggtgtacagcaaggagtgtcaaattccactgggaatggcttctggacgcattagagattttcagattacagcttcaggacaatatggacagtgggccccaaagctggccagacttcattattctggatcaatcaatgcctggagcaccaaggatccctctccttggatcaaggtggatctgttggcgccgatgattattcacagcatcctgactcagggtgcccggcagaagttctccagcctgtacatctctcagtttatcatcatgtacagcctcgatggacagcggtggcagggttatcgggggaactccactgggactttaatggtgttctttggcaatgtggattcatctggagtaaaacacaatatttttaaccctccaattattgctagatatatccgtttgcacccaacgcattacagcatccgcagcactcttcgcatggagttgatgggctgtgacttaaatagttgcaacatgccactgggaatggagaataaagctatatcagatacacagattactgcctcatcccacttaagcaacatgtttgccacctggtctccttcacaagcccgacttaaccttcaagggaggacaaatgcctggagaccccaggtgaataatccaaaagagtggctacaagtggacttccagaagacaatgagagttacaggaataaccactcaaggggtgaaatctctgcttaccagcatgtatgtgaaggagttccttatatccagtagtcaagagggccataactggactccatttcttcagaatggcaaagtgaaggtttttcagggaaatcaagactccttcacccccgtggtgaatactctagaccccccactgtttacccgcttccttcggattcacccgcagagctgggtgcaccatatcgccctgaggctggagttttggggttgtgaggcacagcagcagtactag

(19) United States
(12) Patent Application Publication
(10) Pub. No.: US 2026/0248857 A1
(43) Pub. Date: Aug. 27, 2026

(54) Post-Natal Transplantation Of Factor VIII-Expressing Cells For Treatment of Hemophilia

(71) Applicants: Wake Forest University Health Sciences, Winston-Salem, NC (US); Emory University, Atlanta, GA (US); Children's Healthcare of Atlanta, Inc., Atlanta, GA (US)

(72) Inventors: Maria Graca N.D. Almeida-Porada, Winston-Salem, NC (US); Christopher D. Porada, Winston-Salem, NC (US); Anthony Atala, Winston-Salem, NC (US); Christopher B. Doering, Atlanta, GA (US); H. Trent Spencer, Marietta, GA (US)

(21) Appl. No.: 19/670,484

(22) Filed: May 7, 2026

Related U.S. Application Data
(63) Continuation of application No. 18/129,398, filed on Mar. 31, 2023, now Pat. No. 12,648,967.
Application No. 18/129,398 is a continuation of application No. 16/638,379, filed on Aug. 23, 2018, now abandoned.
(60) Provisional application No. 62/549,280, filed on Aug. 23, 2017.

(51) Int. Cl. A61K 35/28; A61K 38/37; C12N 5/0775

(52) U.S. Cl. CPC A61K 35/28; A61K 38/37; C12N 5/0663; C12N 5/0668; C12N 2510/00

(57) ABSTRACT

Disclosed herein are method of treating hemophilia A in a subject comprising injecting the subject with mesenchymal stromal/stem cells (MSC) modified to express high levels of Factor VIII protein. The MSC are isolated prenatally, at birth, or after the subject's birth. The modified MSC may also express high levels von Willebrand factor protein.

### CROSS REFERENCE TO RELATED APPLICATION

This application is a continuation of U.S. Nonprovisional application Ser. No. 18/129,398, filed Mar. 31, 2023, which is a continuation of U.S. Nonprovisional application Ser. No. 16/638,379, filed Feb. 11, 2020, now abandoned, which is a 371 National Stage of PCT Application No. PCT/US2018/047747, filed Aug. 23, 2018, which claims the benefit of U.S. Provisional Application Ser. No. 62/549,280, filed Aug. 23, 2017, and the contents of these applications are herein incorporated by reference in their entirety.

### STATEMENT AS TO RIGHTS TO INVENTIONS MADE UNDER FEDERALLY SPONSORED RESEARCH AND DEVELOPMENT

This invention was made with government support under grant number 1R01HL130856-01A1 awarded by the U.S. National Institutes of Health (NIH). The government has certain rights in the invention.

### REFERENCE TO A SEQUENCE LISTING SUBMITTED AS AN XML FILE VIA PATENT CENTER

The official copy of the sequence listing is submitted electronically via Patent Center as an XML formatted sequence listing with a file named SEQWFIRM17908.xml, created on May 7, 2026, and has a size of 134,308 bytes and is filed concurrently with the specification. The sequence listing contained in this XML formatted document is part of the specification and is herein incorporated by reference in its entirety.

### BACKGROUND

Factor VIII is an essential blood clotting factor. The protein circulates in the bloodstream in an inactive form, bound to another molecule called von Willebrand factor, until an injury that damages blood vessels occurs. In response to injury, coagulation factor VIII is activated and separates from von Willebrand factor. The active protein interacts with another coagulation factor called Factor IX. This interaction sets off a chain of additional chemical reactions that form a blood clot.

Hemophilia A (HA) is the most common inheritable coagulation deficiency, affecting 1 in 5000 boys, approximately 60% of whom present with the severe form of the disease. Mutations in the Factor VIII gene that result in decreased or defective Factor VIII protein give rise to HA, a recessive X-linked disorder. Individuals with severe HA experience recurrent hematomas of subcutaneous connective tissue/muscle, internal bleeding, and frequent hemarthrosis, leading to chronic debilitating arthropathies. Current treatment is frequent infusions of Factor VIII (plasma-derived or recombinant) to maintain hemostasis, which greatly improves quality of life for many HA patients. While current therapeutic products for HA offer reliable prophylactic and therapeutic efficacy, they are very expensive and do not cure the underlying disease, thus requiring administration for the entire life of the patient. In addition, more than 30% of patients with severe HA develop inhibitory antibodies to the infused Factor VIII therapeutic, placing them in danger of treatment failure. This is a significant and serious complication/challenge in the clinical management/treatment of HA. While protein-based immune tolerance induction (ITI) therapy has been used with some success in this patient group, its cost extends into the millions of dollars per patient, it is only effective in about 60% of patients, and its mechanism of action is largely unknown. These shortcomings with existing therapy for patients who develop inhibitors highlight the need for innovative approaches to surmount this immunological hurdle.

### BRIEF SUMMARY

In one aspect, provided are methods of treating a subject diagnosed with hemophilia A, the method involving the steps of (a) modifying mesenchymal stem/stromal cells (MSC) to express high levels of Factor VIII protein thereby generating modified MSC, the MSC comprising bone-marrow MSC isolated from the subject; (b) generating an expanded modified MSC population by in vitro culturing the modified MSC; and (c) injecting MSC from the expanded modified MSC population into the subject.

In another aspect, provided are methods of treating a subject prenatally diagnosed as having hemophilia A, the method involving the steps of (a) modifying mesenchymal stem/stromal cells (MSC) to express high levels of Factor VIII protein thereby generating modified MSC, the MSC comprising MSC isolated from at least one of amniotic fluid, placental tissue, or umbilical cord tissue obtained at the time of the subject's birth or prenatally from the subject's mother; (b) generating an expanded modified MSC population by in vitro culturing the modified MSC; and (c) injecting MSC from the expanded modified MSC population into the subject.

The above described and many other features and attendant advantages of embodiments of the present disclosure will become apparent and further understood by reference to the following detailed description when considered in conjunction with the accompanying drawings.

### BRIEF DESCRIPTION OF THE DRAWINGS

These figures are intended to be illustrative, not limiting. Although the aspects of the disclosure are generally described in the context of these figures, it should be understood that it is not intended to limit the scope of the disclosure to these particular aspects.

FIG. 1 shows the complete absence of any FVIII antigen/cross-reactive material (CRM) in the hemophilia A sheep according to some aspects of the disclosure.

FIG. 2 shows assessment of phenotypic markers in MSC isolated from sheep according to some aspects of the disclosure.

FIG. 3 shows a schematic diagram of an ovine Factor VIII transgene expression vector according to some aspects of the disclosure.

FIG. 4 shows a schematic diagram of an ovine vWF transgene expression vector according to some aspects of the disclosure.

FIG. 5 shows endogenous expression of Factor VIII in MSC isolated from amniotic fluid according to some aspects of the disclosure.

FIG. 6 shows endogenous expression of vWF in MSC isolated from amniotic fluid according to some aspects of the disclosure.

FIG. 7 shows exogenous expression of Factor VIII in MSC isolated from amniotic fluid and transduced with a lentivector encoding Factor VIII according to some aspects of the disclosure.

FIG. 8A shows assessment of phenotypic markers in PLCs according to some aspects of the disclosure.

FIG. 8B shows flow cytometric analysis of PLC constitutively expressed levels of FVIII protein according to some aspects of the disclosure.

FIG. 8C shows assessment of normalized levels of PLC constitutively expressed levels of FVIII protein according to some aspects of the disclosure.

FIG. 9A shows assessment of normalized levels of secretion of FVIII protein by PLC engineered/transduced to express high levels of FVIII according to some aspects of the disclosure.

FIG. 9B shows assessment of secretion of FVIII protein by transduced as compared to non-transduced PLCs according to some aspects of the disclosure.

FIGS. 10A-10C show assessment of phenotypic markers in transduced as compared to non-transduced PLCs according to some aspects of the disclosure.

FIG. 11A shows assessment of expression of Toll-like receptors (TLRs) in mcoET3-transduced PLCs as compared to non-transduced PLCs according to some aspects of the disclosure.

FIG. 11B shows assessment of expression of stress molecules in mcoET3-transduced PLCs as compared to non-transduced PLCs according to some aspects of the disclosure.

FIG. 12 shows assessment of normalized levels of PLC secretion of FVIII protein by non-transduced-PLC, IcoHSQ-PLC, IcoET3-PLC, ET3-PLC, and mcoET3-PLC according to some aspects of the disclosure.

### DETAILED DESCRIPTION

Provided in this disclosure are methods of treatment for subjects having hemophilia A. The methods are post-natal therapies comprising administering to a subject with hemophilia A autologous mesenchymal stem/stromal cells (MSC) that have been modified to express Factor VIII. Provided methods are effective as first-line therapies for subjects that have been diagnosed prenatally or at an early age and who have not received Factor VIII therapy. Provided methods are also effective as second-line therapies for the treatment of subjects that have been receiving Factor VIII therapy and, in some instances, have developed an immune response to standard infusion therapy of exogenous Factor VIII. The MSC used in the methods are isolated from biological samples obtained prenatally or after the subject's birth. The MSC are modified to express high levels of Factor VIII protein. In some instances, the MSC are modified to express high levels of Factor VIII and high levels of another protein, such as von Willebrand factor. The MSC may be modified by the introduction of a transgene (for example, using a viral vector) or via genome-editing (for example, using the CRISPR/Cas9 system). Administering the modified MSC to the subject results in engraftment of the modified cells. The engrafted cells produce Factor VIII on a continuing basis in the subject and provide long-lasting (ideally lifelong) therapeutic benefit to the subject by promoting blood coagulation.

In one aspect, provided is a method of treating a subject diagnosed clinically or genetically with hemophilia A comprising: (a) modifying mesenchymal stem/stromal cells (MSC) to express high levels of Factor VIII protein thereby generating modified MSC, the MSC comprising bone marrow MSC isolated from the subject; (b) generating an expanded modified MSC population by in vitro culturing the modified MSC; and (c) injecting MSC from the expanded modified MSC population into the subject. The bone marrow MSC express at least one of Stro-1 or CD146. In some instances, the bone marrow MSC may be isolated based on expression of at least one of Stro-1 or CD146.

In another aspect, provided is a method of treating a subject prenatally diagnosed as having hemophilia A comprising: (a) modifying mesenchymal stem/stromal cells (MSC) to express high levels of Factor VIII protein thereby generating modified MSC, the MSC comprising MSC isolated from at least one of amniotic fluid, placental tissue, or umbilical cord tissue obtained at the time of the subject's birth or prenatally; (b) generating an expanded modified MSC population by in vitro culturing the modified MSC; and (c) injecting MSC from the expanded modified MSC population into the subject. In some instances, the MSC are amniotic fluid MSC. In some instances, the MSC are placental MSC (PLC). In some instances, the MSC are umbilical cord tissue MSC.

As used herein the terms treatment, treat, or treating refer to a method of reducing one or more symptoms of a disease or condition. In some instances, treatment results in a 5%, 10%, 20%, 30%, 40%, 50%, 60%, 70%, 80%, 90%, or 100% reduction in the severity of one or more symptoms of the disease or condition. In some instances, treatment results in at least a 60%, 65%, 70%, 75%, 80%, 85%, 90%, or 95% reduction in the severity of one or more symptoms of the disease or condition. In some instances, treatment results in a 100% reduction in the severity of one or more symptoms of the disease or condition. For example, a method for treating a disease is considered to be a treatment if there is a 5% reduction in one or more symptoms or signs. As used herein, control refers to the untreated condition. In some instances, the reduction can be a 5%, 10%, 20%, 30%, 40%, 50%, 60%, 70%, 80%, 90%, 100%, or any percent reduction in between 10% and 100% as compared to native or control levels. In some instances, the reduction can be at least a 65%, 70%, 75%, 80%, 85%, 90%, or 95% reduction as compared to native or control levels. In some instances, the reduction can be a 100% reduction. It is understood that treatment does not necessarily refer to a cure or complete ablation of the disease, condition, or symptoms of the disease or condition. As used herein, references to decreasing, reducing, or inhibiting include a change of 5%, 10%, 20%, 30%, 40%, 50%, 60%, 70%, 80%, 90% or greater as compared to a control level. Such terms can include, but do not necessarily include, complete elimination.

The subject on which the method is performed has been diagnosed with hemophilia A. The subject is mammalian, including humans; non-human primates, such as apes and monkeys; cattle; horses; sheep; rats; dogs; cats; mice; pigs; and goats. In some embodiments, the subject is a human, a dog, a horse, a sheep, a cow, or a cat. The subject may be male or female. The subject may be a juvenile subject or an adult subject. As a recessive X-linked disorder, a male subject will carry an X chromosome that has a mutation in the Factor VIII gene. A female subject that has hemophilia A will either have a mutated Factor VIII allele on both X chromosomes or will have a mutant Factor VIII allele on one X chromosome and have an inactive Factor VIII allele on the other X chromosome. In some instances, the subject may be a female carrier of hemophilia A that has a mutant Factor VIII allele on one X chromosome and a normal Factor VIII gene on the other X chromosome. Subjects may be diagnosed via prenatal genetic testing, particularly in instances where there is a family history of hemophilia. The DNA from biological samples obtained from amniocentesis, chorionic villi sampling, or cell-free fetal DNA present in the maternal peripheral blood may be analyzed for abnormalities on the X chromosome or mutations in the Factor VIII gene. Alternatively, subjects may be diagnosed after birth by assessing the ability of the subject's blood to clot properly. For example, screening tests include activated partial thromboplastin time (APTT) test, prothrombin time (PT) test, and fibrinogen test. Diagnosis of hemophilia A (type and severity) can also be performed with antigen-based tests that assess the amount of Factor VIII protein in the subject's blood. In some instances, the subject may have received therapy with infused Factor VIII protein. Where the subject has received such therapy, in some instances the subject may have developed an immune response to Factor VIII protein (developed inhibitory antibodies that impair the effectiveness of the therapy). In some embodiments, the subject has received prior treatment with exogenous Factor VIII and has developed an inhibitory immune response that diminishes the effectiveness of the exogenous Factor VIII treatment.

MSC, referred to in the field as mesenchymal stem cells, mesenchymal stromal cells, and, when isolated from bone marrow, also marrow stromal progenitors (MSP), are multipotent stromal cells that can differentiate into a variety of cell types, including: osteoblasts, chondrocytes, myocytes, and adipocytes. MSC do not have the capacity to reconstitute an entire organ. The term encompasses multipotent cells derived from other non-marrow tissues, such as placenta, umbilical cord blood, adipose tissue, or the dental pulp of deciduous baby teeth. MSC are heterogeneous and different subsets of MSC may have different capabilities. Different methods of isolation will result in different populations of MSC. Such different populations may express different protein markers. MSC subpopulations with different marker expression profiles have been found to have different capabilities. See, for example, Thierry, D., et al., Stro-1 Positive and Stro-1 Negative Human Mesenchymal Stem Cells Express Different Levels of Immunosuppression, Blood 104(11): 4964 (2004). The extent to which a MSC population isolated using one method and having a particular marker profile will share properties with a MSC population isolated using a different method and having a different marker profile has not been determined.

In some instances, the MSC used in the method are bone marrow-derived MSC—that is, the MSC are isolated from bone marrow. Specifically, the bone marrow-derived MSC are isolated from bone marrow obtained from the subject (autologous MSC). In some instances, the bone marrow-derived MSC used in the method express Stro-1, CD146, or both Stro-1 and CD146. Flow cytometry methods may be used to isolate MSC expressing these markers such as described, for example, in Sanada C., et al., Mesenchymal stem cells contribute to endogenous FVIII: c production. J Cell Physiol. 2013; 228 (5): 1010-1016 and Chamberlain J. L., et al., Efficient generation of human hepatocytes by the intrahepatic delivery of clonal human mesenchymal stem cells in fetal sheep. Hepatology. 2007; 46(6): 1935-1945. Isolating MSC based on Stro-1 and/or CD146 results in a distinct cell population from that isolated using the traditional approach in which bulk unpurified bone marrow or Ficoll-purified bone marrow mononuclear cells are plated directly into plastic cell culture plates or flasks to which the adherent MSC population binds.

In some instances, the MSC used in the method are MSC isolated from a birth tissue or birth fluid. Specifically, the MSC may be isolated from amniotic fluid, placental tissue, chorionic villi, or umbilical cord tissue. In some instances, the MSC used in the method express c-kit. Methods of isolating such cells are described in U.S. Pat. Nos. 7,968,336 and 8,021,876, which are incorporated herein by reference in their entirety. In some instances, the MSC express at least one of c-kit, CD34, CD90, or CD133. In some instances, the MSC express c-kit and at least one of CD34, CD90, or CD133. In some instances, the MSC are isolated based on expression of c-kit.

For juvenile patients for whom prenatal biological samples are available, the MSC may be isolated from such samples (such as amniotic fluid, placental, cord tissue). Such samples may be available where the subject is diagnosed with hemophilia prior to birth. In some instances, appropriate biological samples may be obtained at the time of the subject's birth (such as amniotic fluid, placental, cord tissue). For adult patients, or juvenile patients for which prenatal biological samples are not available, the MSC used in the method may be bone marrow derived mesenchymal stem/stromal cells (MSC), also referred to as bone marrow stromal cells.

The MSC used in the method are modified to express high levels of Factor VIII. In some instances, the MSC may be modified to also express high levels of von Willebrand factor (vWF).

In some instances, an exogenous gene sequence encoding one or both of these proteins may be introduced into the MSC via one or more vectors. In some instances, the MSC may be modified to express high levels of Factor VIII protein via introduction into the MSC of a vector comprising a Factor VIII gene sequence operatively linked to a constitutively active promoter. In some instances, the MSC may be modified to express high levels of vWF protein via introduction into the MSC of a vector comprising a vWF gene sequence operatively linked to a constitutively active promoter. In some instances, the MSC may be modified to express high levels of Factor VIII protein and vWF protein via introduction into the MSC of a vector comprising a Factor VIII gene sequence operatively linked to a constitutively active promoter and a vector encoding a vWF gene sequence operatively linked to a constitutively active promoter. In some instances, the Factor VIII gene sequence and the vWF gene sequence may be operatively linked to the same constitutively active promoter. Alternatively, the Factor VIII gene sequence and the vWF gene sequence may be operatively linked to different constitutively active promoters.

Exemplary vectors include, for example, plasmids and viral vectors (including but not limited to adenoviral, adeno-associated viral (AAV), or retroviruses such as lentiviruses. In preferred embodiments, the vector is a viral vector. In some instances, the vector may be a vector that integrates into the genome of transduced cells. For example, the vector may be a lentivirus vector. In preferred embodiments, the vector is a lentivirus vector. In some instances, the lentivirus vector contains a 3′-modified long terminal repeat (LTR), resulting in a self-inactivating (SIN) lentivector. A lentivirus vector may integrate into the genome of dividing or non-dividing cells. The lentivirus genome in the form of RNA is reverse-transcribed to DNA when the virus enters the cell, and is then inserted into the genome by the viral integrase enzyme. The lentivirus vector, now called a provirus, remains in the genome and is passed on to the progeny of the cell when it divides. In another example, the vector may be an adeno-associated virus (AAV) vector, which, in contrast to wild-type AAV, only rarely integrates into the genome of the cells it transduces. In one example, the vector may be an adenoviral vector. An adenoviral vector does not integrate into the genome. In another instance, the vector may be a murine retrovirus vector. In another example, the vector may be a foamy virus vector, which may have a larger capacity for inserts than lentiviral vectors. In another example, the vector may be Sendai virus vector.

The exogenous gene sequences are operatively linked to one or more promoter sequences within the vector. The term “promoter sequence” or “promoter element” refers to a nucleotide sequence that assists with controlling expression of a coding sequence. Generally, promoter elements are located 5′ of the translation start site of a gene. However, in certain embodiments, a promoter element may be located within an intron sequence, or 3′ of the coding sequence. In some embodiments, a promoter useful for a gene therapy vector is derived from the native gene of the target protein (e.g., a Factor VIII promoter). In some embodiments, the promoter is a constitutive promoter, which drives substantially constant expression of protein from the exogenous gene sequence. Non-limiting examples of well-characterized promoter elements include the cytomegalovirus immediate-early promoter (CMV), the β-actin promoter, the methyl CpG binding protein 2 (MeCP2) promoter, the simian virus 40 early (SV40) promoter, human Ubiquitin C promoter (UBC), human elongation factor 1α promoter (EF1α), the phosphoglycerate kinase 1 promoter (PGK), or the CMV immediate early enhancer/chicken beta actin (CAG) promoter. The vector will generally also contain one or more of a promoter regulatory region (e.g., one conferring constitutive expression), a transcription initiation start site, a ribosome binding site, an RNA processing signal, a transcription termination site, and/or a polyadenylation signal.

In some instances, the Factor VIII transgene is operably linked to a promoter. A number of promoters can be used in the practice of the invention. The promoters can be selected based on desired outcome. The nucleic acids can be combined with constitutive, inducible, tissue-preferred, or other promoters for expression in the organism of interest. See, for example, promoters set forth as SEQ ID NOs: 1-6 as described in Brown et al. (2018) Target-Cell-Directed Bioengineering Approaches for Gene Therapy of Hemophilia A. Mol. Ther. Methods Clin. Dev., 2018. 9:57-69, which is herein incorporated by reference in its entirety for all purposes.

Where the MSC are modified to express high levels of Factor VIII via transduction with an exogenous Factor VIII gene sequence, the exogenous Factor VIII gene sequence may be human Factor VIII gene sequence, porcine Factor VIII gene sequence, or a hybrid transgene comprising portions of human Factor VIII gene sequence and portions of porcine Factor VIII gene sequence. In some instances, the gene sequence comprises all or a portion of the human Factor cDNA as set forth in GenBank Accession No. 192448441 as updated Jul. 17, 2017, wherein said portion would encode a function portion of the human Factor VIII protein. In some instances, the gene sequence comprises a sequence that is at least 99%, 98%, 97%, 96%, 95%, 94%, 93%, 92%, 90%, 89%, 88%, 87%, 86%, 85%, 84%, 83%, 82%, 81%, or 80% identical to all or a portion of the human Factor cDNA as set forth in GenBank Accession No. 192448441 as updated Jul. 17, 2017, wherein said portion would encode a functional portion of the human Factor VIII protein. In some instances, the gene sequence may encode all or a functional portion of the human Factor VIII protein as set forth in GenBank Accession No. 192448441 as updated Jul. 17, 2017, reflecting the protein transcribed from transcript variant 1 of the Factor VIII gene. This protein is approximately 300 kDa and contains a series of homology-defined domains designated A1-A2-B-ap-A3-C₁-C₂. In some instances, the exogenous Factor VIII gene sequence is modified relative to wild-type protein sequence to result in increased protein expression, increased protein stability, reduced immunogenicity, or a combination of one or more thereof.

In some instances, the sequence of one or more of the Factor VIII protein domains may be deleted. In one example, the B domain of Factor VIII is deleted. The B domain of Factor VIII has no known function and can be deleted without loss of coagulant activity. Deletion of the B-domain has been shown to increase factor VIII protein production in heterologous systems (Toole et al. (1986) Proc. Natl. Acad. Sci. U.S.A. 83:5939-5942). In addition, wildtype porcine Factor VIII protein having the B-domain deleted may have 10-100-fold higher expression and secretion than the human Factor FVIII gene sequence, both in vitro and in vivo. (See, for example, Dooriss, K. L., et al., Comparison of factor VIII transgenes bioengineered for improved expression in gene therapy of hemophilia A. Hum Gene Ther. 20:465-478 (2009). A B-domain deleted form of human Factor VIII protein (Lind et al. (1995) Eur. J. Biochem. 232:19-27) has been approved for clinical use.

In some instances, the exogenous Factor VIII gene sequence may include protein modifications to reduce immunogenicity of the protein thereby reducing the risk of an immune response due to therapy. For example, alanine substitutions may be included as described in Healey, J. F., et al., The comparative immunogenicity of human and porcine factor VIII in haemophilia A mice. Thromb Haemost. 102:35-41 (2009) and Lubin, I. M., et al., Analysis of the human factor VIII A2 inhibitor epitope by alanine scanning mutagenesis. J Biol Chem. 272:30191-30195 (1997), which are incorporated by reference herein in their entirety.

In some instances, one or more of the human Factor VIII protein domain sequences may be substituted with the sequence of the corresponding porcine Factor VIII protein domain sequences. For example, one or more porcine Factor VIII domains may be substituted for one or more human Factor VIII domains. For example, inclusion of the porcine Factor VIII domains A1 and ap-A3 may increase expression of the expressed Factor VIII protein. See, for example, Doering, C. B., et al., Identification of porcine coagulation factor VIII domains responsible for high level expression via enhanced secretion. J Biol Chem. 279:6546-6552 (2004). In some embodiments, the exogenous Factor VIII gene sequence may comprise the human Factor VIII A2 and C2 domains and the porcine Factor VIII A1, A3, and C1 domains.

In some instances, the exogenous Factor VIII gene sequence may comprise a modified Factor VIII sequence comprising a B domain-deleted (BDD) Factor VIII transgene having the sequence of the human A2 and C2 domains and the porcine A1, A3, and C1 domains, and also include three alanine substitutions in the A2 domain to reduce immunogenicity, as described in Lubin, I. M., et al., Analysis of the human factor VIII A2 inhibitor epitope by alanine scanning mutagenesis. J Biol Chem. 1997; 272(48):30191-5. This modified Factor VIII protein is referred to as the ET3 transgene in this disclosure, including in the Examples below. In some instances, the ET3 transgene is expressed at a comparable level to that of wild-type porcine Factor VIII protein while having 91% identity to the amino acid sequence of wild-type human Factor VIII protein. In one example, the exogenous Factor VIII gene sequence may comprise a human/porcine Factor VIII transgene as described in Doering, C. B., et al., Directed engineering of a high-expression chimeric transgene as a strategy for gene therapy of hemophilia A, Mol. Ther. 17(7):1145-1154 (2009), which is incorporated herein by reference in its entirety.

In some instances, the Factor VIII transgene sequence may comprise one of the modified Factor VIII sequences described in Brown et al. (2018) Target-Cell-Directed Bioengineering Approaches for Gene Therapy of Hemophilia A. Mol. Ther. Methods Clin. Dev., 2018. 9:57-69, which is incorporated herein by reference in its entirety for all purposes. Factor VIII polypeptides, including tissue-specific codon optimized variants, are described therein. Modified Factor VIII transgene sequences used in the methods described herein can be any one of SEQ ID NOs: 7-16 (as described in Brown et al.). For example, Factor VIII transgene sequences that can be used in the methods described herein include a B-domain deleted (BDD) human Factor VIII polypeptide (HSQ) as set forth in SEQ ID NO: 15, a BDD chimeric human/porcine Factor VIII polypeptide (ET3) as set forth in SEQ ID NO: 11, or an ancestral Factor VIII polypeptide (An53) as set forth in SEQ ID NO: 7.

In some instances, the exogenous Factor VIII gene sequence may be modified for expression in a particular organ or tissue type. For example, the gene sequence may be optimized for expression in myeloid tissue. In some embodiments, the Factor VIII transgene may comprise myeloid codon optimized ET3 (mcoET3) as set forth in SEQ ID NO: 12 or myeloid codon optimized HSQ (mcoHSQ) as set forth in SEQ ID NO: 16. Alternatively, the Factor VIII transgene may be optimized for expression in liver tissue. In some embodiments, the Factor VIII transgene may comprise liver codon optimized ET3 (lcoET3) as set forth in SEQ ID NO: 10; liver codon optimized An53 as set forth in SEQ ID NO: 8; or liver codon optimized (lcoHSQ) as set forth in SEQ ID NO: 14.

In some instances, the exogenous Factor VIII gene sequence may comprise one of the modified Factor VIII sequences described in U.S. Pat. No. 7,635,763, which is incorporated herein by reference in its entirety for all purposes. Regions of the porcine Factor VIII polypeptide that comprises the A1 and ap-A3 regions, and variants and fragments thereof, are described therein that impart high-level expression to both the porcine and human Factor VIII polypeptide. The exogenous Factor VIII gene sequence encoded by the viral vector of the provided methods may be the polynucleotides set forth in any one of SEQ ID NOs: 19, 21, 23, 25, or 27 (SEQ ID NOs: 15, 17, 19, 13, or 21 as described in U.S. Pat. No. 7,635,763). The modified Factor VIII protein expressed at high levels in the modified MSC may comprise the amino acid sequences set forth in any one of SEQ ID NOs: 18, 20, 22, 24, or 26 (SEQ ID NOs: 14, 16, 18, 12, or 20 as described in U.S. Pat. No. 7,635,763). Such sequences are summarized in Table 1 below. In some instances, these sequences may be used to construct an exogenous Factor VIII gene sequence encoding a modified factor VIII polypeptide that results in a high level of expression of the encoded modified Factor VIII protein.

TABLE 1
Exemplary Modified Factor VIII Proteins

| Modified Factor | | |
|---|---|---|
| VIII Protein | SEQ ID NO. | Description |
| HP44/OL | aa: SEQ ID NO: 18 | A1_{P}-A2_{P}-ap_{P}-A3_{P}-C1_{H}-C2_{H} |
| | nt: SEQ ID NO: 19 | porcine A1, A2, ap-A3 domains, |
| | | porcine-derived linker sequence |
| | | S F A Q N S R P P S A S A P K |
| | | P P V L R R H Q R (SEQ |
| | | ID NO: 30), and human C1 and C2 |
| | | domains |
| HP46/SQ | aa: SEQ ID NO: 20 | A1_{P}-A2_{H}-ap_{H}-A3_{H}-C1_{H}-C2_{H} |
| | nt: SEQ ID NO: 21 | porcine A1 domain, human A2, |
| | | ap-A3, C1 and C2 domains, |
| | | and human S F S Q |
| | | N P P V L K R H Q R |
| | | linker sequence |
| HP47/OL | aa: SEQ ID NO: 22 | A1_{P}-A2_{H}-apₚ-A3_{P}-C1_{H}-C2_{H} |
| | nt: SEQ ID NO: 23 | porcine A1, ap-A3 domains, |
| | | porcine-derived linker sequence |
| | | S F A Q N S R P P S A S A P K |
| | | P P V L R R H Q R (SEQ |
| | | ID NO: 31), and human A2, C1 |
| | | and C2 domains |
| B-domain | aa: SEQ ID NO: 24 | Human Factor VIII protein sequence |
| deleted | nt: SEQ ID NO: 25 | minus B-domain |
| HP63/OL | aa: SEQ ID NO: 26 | porcine A1 domain and a |
| | nt: SEQ ID NO: 27 | partially humanized ap-A3 domain |
| | | that comprises porcine amino acids |
| | | from about 1690 to about 1804 |
| | | and from about 1819 to about 2019 |

As discussed above, in some instances, the MSC are also modified to express high levels of vWF protein via introduction into the MSC of a vector. In some embodiments coding sequences for vWF can be any one of SEQ ID NOs: 28 or 29. In some instances, the vWF gene sequence in the vector may encode all or a functional portion of the human vWF protein set forth in GenBank Accession No. 1023301060 as updated Aug. 21, 2017. However, in some instances, the vWF gene sequence may include one or more modifications to the wild-type vWF gene sequence to increase protein expression, increase protein stability, reduce immunogenicity, or a combination of one or more thereof, of the vWF protein. For example, the full cDNA sequence of the vWF gene may be too large to be packaged efficiently in certain vectors, such as, for example, a lentiviral vector. Thus, in some instances, one or more exons of the vWF gene may be deleted while still retaining biological function of the expressed protein. In some instances, exons 24-46 of the vWF gene may be deleted as described in U.S. Patent Application Publication No. 2010/0183556. In some instances, the vWF gene sequence may be codon-optimized for efficient expression in the MSC. In some instances, the exogenous vWF gene sequence may modified for expression in a particular organ or tissue type. For example, the gene sequence may be optimized for expression in the liver. Thus, in some instances, the vWF gene sequence may comprise at least 99%, 98%, 97%, 96%, 95%, 94%, 93%, 92%, 90%, 89% 88%, 87%, 86%, 85%, 84%, 83%, 82%, 81%, or 80% identity to the corresponding wild-type vWF gene sequence and comprise modifications to improve expression. In some instances, the vWF gene sequence comprises the truncated human vWF sequence set forth below in this disclosure or a sequence at least 99%, 98%, 97%, 96%, 95%, 94%, 93%, 92%, 90%, 89% 88%, 87%, 86%, 85%, 84%, 83%, 82%, 81%, or 80% identical thereto while retaining biological activity of the expressed protein. In some instances, the vWF gene sequence comprises the truncated sheep vWF sequence set forth below in this disclosure or a sequence at least 99%, 98%, 97%, 96%, 95%, 94%, 93%, 92%, 90%, 89% 88%, 87%, 86%, 85%, 84%, 83%, 82%, 81%, or 80% identical thereto while retaining biological activity of the expressed protein.

In some instances, gene-editing may be performed on the MSC to insert, delete, or replace the genomic sequence of one or both of the endogenous genes using engineered nucleases (molecular scissors). Gene-editing nucleases belong to one of three known categories: zinc-finger nucleases (ZFN), transcription activator-like effector nucleases (TALEN), and clustered regularly interspaced short palindromic repeats (CRISPR) and their associated proteins (Cas) tools. All operate on the same principle; they are all capable of inducing a double-strand break at a defined genomic sequence that is subsequently corrected by endogenous DNA repair mechanisms. Double-strand breaks can be repaired through homology-driven repair (HDR), in the presence of donor homologous DNA sequences, resulting in gene-editing events.

In some instances, the MSC may be modified to express high levels of the Factor VIII protein via gene-editing of an endogenous Factor VIII gene sequence of the MSC, wherein the gene-editing introduces one or more modifications to an endogenous Factor VIII gene sequence that increase protein expression, increase protein stability, reduce immunogenicity, or a combination of one or more thereof, of the Factor VIII protein. In some instances, the MSC are modified to express high levels of an exogenous FVIII protein via genome-editing, wherein the gene-editing introduces an exogenous FVIII gene, under the control of a constitutive promoter, into a “safe harbor” region within the genome, such as the AAVS1 site. In some instances, the MSC are modified to express high levels of the vWF protein via gene-editing of an endogenous vWF gene sequence of the MSC, wherein the gene editing introduces one or more modifications to the endogenous vWF gene sequence that increase protein expression, increase protein stability, reduce immunogenicity, or a combination of one or more thereof, of the vWF protein. In some instances, the MSC are modified to express high levels of an exogenous vWF protein via genome-editing, wherein the gene-editing introduces an exogenous vWF gene, under the control of a constitutive promoter, into a “safe harbor” within the genome, such as the AAVS1 site. Exemplary “safe harbor” regions are described in Cerbini, T., et al., Transfection, selection, and colony-picking of human induced pluripotent stem cells TALEN-targeted with a GFP gene into the AAVS1 safe harbor. J Vis Exp. 2015 Feb. 1; (96): 52504 and Hong, S. G., et al., Rhesus iPSC Safe Harbor Gene-Editing Platform for Stable Expression of Transgenes in Differentiated Cells of All Germ Layers. Mol Ther. 2017; 25(1):44-53.

In some instances, the endogenous Factor VIII gene sequence may be modified by gene-editing to have the type of modifications described above for embodiments where an exogenous Factor VIII gene sequence is introduced via transduction. The discussion of the various modifications described above is thus also applicable to embodiments where the endogenous Factor VIII gene sequence is modified. For example, in some instances, the sequence of one or more protein domains of the endogenous Factor VIII gene sequence may be deleted. In some instances, the B domain of Factor VIII is deleted. In some instances, the endogenous Factor VIII gene sequence may be modified to reduce immunogenicity of the protein thereby reducing the risk of an immune response due to therapy. For example, alanine substitutions may be introduced as described in Healey, J. F., et al., The comparative immunogenicity of human and porcine factor VIII in haemophilia A mice. Thromb Haemost. 102:35-41 (2009) and Lubin, I. M., et al., Analysis of the human factor VIII A2 inhibitor epitope by alanine scanning mutagenesis. J Biol Chem. 272:30191-30195 (1997), which are incorporated by reference herein in their entirety.

In some instances, the endogenous Factor VIII gene sequence may be modified to substitute one or more of the Factor VIII protein domain sequences with the sequence of the corresponding Factor VIII protein domain sequences from another species. For example, for human subjects, the endogenous Factor VIII gene sequence may be modified to substitute one or more of the human Factor VIII protein domain sequences with the sequence of the corresponding porcine Factor VIII protein domain sequences. For example, substitution with the porcine Factor VIII domains A1 and ap-A3 may increase expression of the expressed Factor VIII protein. See, for example, Doering, C. B., et al., Identification of porcine coagulation factor VIII domains responsible for high level expression via enhanced secretion. J Biol Chem. 279:6546-6552 (2004). In some embodiments, the endogenous Factor VIII gene sequence may be modified to comprise the porcine Factor VIII A1, A3, and C1 domains, while retaining the human Factor VIII A2 and C2 domains.

In some instances, the endogenous Factor VIII gene sequence may be modified to include a B domain deletion, the porcine A1, A3, and C1 domains, and also include three alanine substitutions in the A2 domain to reduce immunogenicity, as described above for the exogenous Factor VIII gene sequence embodiments. In one example, the endogenous Factor VIII gene sequence may be modified to have the sequence of a human/porcine Factor VIII transgene as described in Doering, C. B., et al., Directed engineering of a high-expression chimeric transgene as a strategy for gene therapy of hemophilia A, Mol. Ther. 17(7):1145-1154 (2009), which is incorporated herein by reference in its entirety. In some instances, the modified endogenous Factor VIII gene sequence results in expression of a modified Factor VIII protein at a level comparable to that of wild-type porcine Factor VIII protein while having 91% identity to the amino acid sequence of wild-type human Factor VIII protein.

In some instances, the endogenous Factor VIII gene sequence may be modified to comprise one of the modified Factor VIII sequences described in U.S. Pat. No. 7,635,763, which is incorporated herein by reference in its entirety for all purposes. In some instances, the endogenous Factor VIII gene sequence may comprise the polynucleotides set forth in any one of SEQ ID NOs: 19, 21, 23, 25, or 27 (SEQ ID NOs: 15, 17, 19, 13, or 21 as described in U.S. Pat. No. 7,635,763). The modified Factor VIII protein expressed at high levels in the modified MSC may comprise the amino acid sequences set forth in any one of SEQ ID NOs: 18, 20, 22, 24, or 26 (SEQ ID NOs: 14, 16, 18, 12, or 20 as described in U.S. Pat. No. 7,635,763). Such sequences are summarized in Table 1 above.

As discussed above, in some instances, the MSC are also modified to express high levels of vWF protein via gene-editing. In some instances, the vWF gene sequence may include one or more modifications to the wild-type vWF gene sequence to increase protein expression, increase protein stability, reduce immunogenicity, or a combination of one or more thereof, of the vWF protein. For example, in some instances, one or more exons of the vWF gene may be deleted while still retaining biological function of the expressed protein. In some instances, exons 24-46 of the vWF gene may be deleted as described in U.S. Patent Application Publication No. 2010/0183556. In some instances, the vWF gene sequence may be codon-optimized for efficient expression in the MSC. In some instances, the exogenous vWF gene sequence may modified for expression in a particular organ or tissue type. For example, the gene sequence may be optimized for expression in the liver. Thus, in some instances, the vWF gene sequence may be modified to comprise at least 99%, 98%, 97%, 96%, 95%, 94%, 93%, 92%, 90%, 89% 88%, 87%, 86%, 85%, 84%, 83%, 82%, 81%, or 80% identity to the corresponding wild-type vWF gene sequence and comprise modifications to improve expression. In some instances, the vWF gene sequence may be modified to comprise the truncated human vWF sequence set forth below in this disclosure or a sequence at least 99%, 98%, 97%, 96%, 95%, 94%, 93%, 92%, 90%, 89% 88%, 87%, 86%, 85%, 84%, 83%, 82%, 81%, or 80% identical thereto while retaining biological activity of the expressed protein. In some instances, the vWF gene sequence may be modified to comprise the truncated sheep vWF sequence set forth below in this disclosure or a sequence at least 99%, 98%, 97%, 96%, 95%, 94%, 93%, 92%, 90%, 89% 88%, 87%, 86%, 85%, 84%, 83%, 82%, 81%, or 80% identical thereto while retaining biological activity of the expressed protein.

Where the MSC are modified to express high levels of both Factor VIII and vWF, the same method of modification may be used to achieve high expression of both proteins or different methods could be used for each protein. For example, in some instances, the MSC may be modified to express high levels of both Factor VIII and vWF protein via introduction of exogenous gene sequences for both proteins. In another example, the MSC may be modified to express high levels of both Factor VIII and vWF protein via gene-editing of the endogenous gene sequences of both proteins. In some instances, the MSC may be modified to express high levels of Factor VIII via transduction of an exogenous Factor VIII gene sequence and modified to express high levels of vWF via gene-editing of the endogenous vWF gene sequences. In other instances, the MSC may be modified to express high levels of vWF via transduction of an exogenous vWF gene sequence and modified to express high levels of Factor VIII via gene-editing of the endogenous Factor VIII gene sequences.

A “high level of expression” means that the production/expression of the modified Factor VIII protein or vWF protein is at an increased level as compared to the expression level of the corresponding native Factor VIII protein or vWF protein expressed under the same conditions. An increase in protein expression levels (considered a high level of expression) comprises at least about 0.5, 1, 2, 3, 4, 5, 6, 7, 8, 9, 10, 11, 12, 13, 14, 15, 20-fold or greater expression of the modified Factor VIII protein or vWF protein compared to the expression levels of the corresponding Factor VIII protein or vWF protein. Alternatively, “high-level expression” can comprise an increase in protein expression levels of at least 1-25 fold, 1-5 fold, 5-10 fold, 10-15 fold, 15-20 fold, 20-25 fold or greater expression levels of the modified Factor VIII protein or vWF protein when compared to the corresponding Factor VIII protein or vWF protein. Methods for assaying protein expression levels are routine in the art. By “corresponding” Factor VIII protein or vWF protein is intended a Factor VIII protein or vWF protein that comprises an equivalent amino acid sequence. In one example, expression of a modified human Factor VIII protein comprising the A1-A2-ap-A3-C₁-C₂ domains is compared to a human Factor VIII protein containing corresponding domains A1-A2-ap-A3-C₁-C₂. In another example, for a fragment of a modified human Factor VIII protein containing domains A1-A2-ap-A3, expression is compared to a fragment of human Factor VIII protein having the corresponding domains A1-A2-ap-A3. Alternatively, in certain instances, expression of a modified Factor VIII protein or vWF protein may be compared to the full-length corresponding proteins. In one example, for a fragment of a modified human Factor VIII protein containing domains A1-A2-ap-A3, expression is compared to human Factor VIII protein having the A1-A2-ap-A3-C1-C2 domains.

The modified MSC are cultured in vitro to generate an expanded modified MSC population. The expanded modified MSC population provides sufficient numbers of modified MSC for therapeutic use. Culture conditions may be selected based on the type of MSC used in the method. For example, MSC isolated from placental tissue may be grown in culture medium optimized for placental cells. In another example, MSC isolated from amnion tissue may be grown in culture medium optimized for amniotic cells. In another example, MSC isolated from umbilical cord or bone marrow may be grown in culture medium optimized for MSC cells. The modified cells may be grown on plastic culture dishes for at least 2, 3, 4, 5, or 6 passages to generate the expanded modified MSC population. In some instances, all or a portion of the expanded modified MSC population may be cryopreserved.

Following culturing of the modified MSC to generate expanded modified MSC population, modified MSC from expanded modified MSC population are injected into the subject. The injection may be made at least one of intraperitoneal injection, intravenous injection, or intra-articular injection. Each injection comprises about 10⁵ to about 10⁹ MSC from the expanded modified MSC population per kilogram weight of the subject. For example, the injection may comprise 10⁵ MSC, 10⁶ MSC, 10⁷ MSC, 10⁸ MSC, or 10⁹ MSC. The number of cells injected into the subject is based on the amount of protein expressed per cell. This metric is determined empirically for the expanded modified MSC population. In some instances, this metric may be generally predictable based on the nature of the modified MSC (for example, method of modification, Factor VIII gene sequence, vWF gene sequence, vector and vector components).

In some instances, modified MSC are injected into the subject once, twice, 3 times, 4 times, 5 times, 6 times, 7 times, 8 times, 9 times, or 10 times. In some instances, modified MSC are injected into the subject at least once, at least twice, at least 3 times, at least 4 times, at least 5 times, at least 6 times, at least 7 times, at least 8 times, at least 9 times, or at least 10 times. For example, the modified MSC may injected as multiple injections on the same day. In some instances, the modified MSC may be injected into the subject on multiple days. In some instances, the subject is injected with modified MSC on a first day and then the subject may be monitored over a period of time (days or weeks) to determine if there is sufficient protein expression to provide the desired therapeutic benefit. In some instances, the amount of protein expression in the subject's blood of Factor VIII protein, vWF protein, or both, may be monitored. In some instances, the efficiency of the subject's blood to clot may be assessed using routine blood clotting tests known in the art. In some instances, the subject's symptoms relating to joint pain and/or inflammation may be assessed. Where monitoring indicates that the amount of expression of Factor VIII protein alone, or the amount of expression of Factor VIII protein and vWF protein, is insufficient, the subject's disease symptoms are not alleviated, or both, the subject may be injected with modified MSC on a second day. Again, the subject may be monitored over a period of time to determine if there is sufficient protein expression to provide the desired therapeutic benefit. These steps may be repeated for a fourth, fifth, sixth, seventh, eighth, ninth, or tenth injection as needed to achieve the desired therapeutic benefit of alleviating the subject's disease symptoms.

In some instances, the use of MSC as cellular vehicles to deliver a Factor VIII gene sequence, a vWF gene sequence, or both, to a subject (as opposed to administration of vector directly) may overcome limitations/risks observed to-date in AAV-based clinical trials for hemophilia: 1) the possibility of off-target transduction of troubling cell types, such as germline cells; 2) the inability to treat patients with pre-existing antibodies to the serotype of AAV being employed as a vector; and 3) the transient hepatotoxicity induced by the AAV capsid, that triggers subsequent immune/inflammatory destruction of many of the transduced cells. Although early studies in vitro and in normal and hemophilia A mice, have used unselected stromal cells (isolated based solely upon plastic adherence) as cellular vehicles for delivering exogenous Factor VIII, no attempts have yet been made to use phenotypically-defined MSC/pericytes to deliver FVIII in vivo in any preclinical model of hemophilia A.

In some instances, the use of MSC as cellular vehicles to deliver a therapeutic gene is also an improvement over the use of hematopoietic stem cells (HSC), as have been used in most cell-based gene therapy trials. The use of MSC eliminates the possibility of insertional leukemogenesis, which is the most serious adverse event seen to-date in clinical gene therapy trials. A successful outcome of the proposed studies targeting hemophilia A thus promises to open the door to safe correction of a variety of congenital disorders using MSC to deliver the therapeutic gene.

### Examples

### Example 1. Animal Model

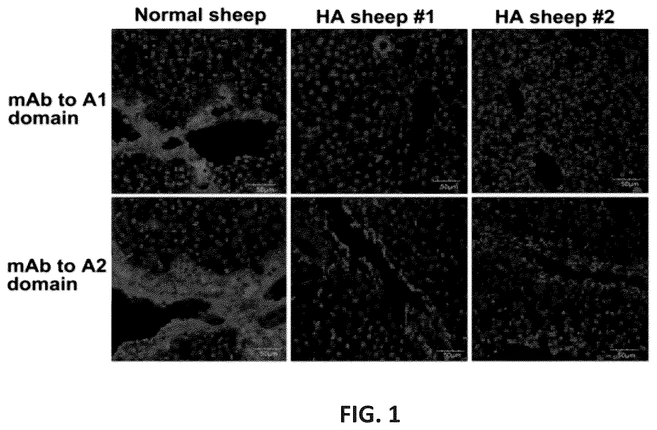

Applicant re-established a line of sheep that emulates the genetics, inhibitor formation, and clinical symptoms of the severe form of human hemophilia A (HA), including the development of frequent, spontaneous hematomas and crippling hemarthroses, making them unique among the HA models. See Porada, C. D., et al., Clinical and molecular characterization of a re-established line of sheep exhibiting hemophilia A. J Thromb Haemost, 2010. 8(2): 276-285. Using unique antibodies developed to various regions of the ovine FVIII protein, it was determined that these sheep do not produce any FVIII antigen (FIG. 1), as demonstrated by the complete lack of any staining within the liver of two of the HA sheep, which is in marked contrast to the widespread bright staining that is seen in the liver from a normal/healthy sheep. As such, they should be an excellent model of severe, cross-reacting material (CRM)-negative hemophilia A patients. Additionally, sheep are close in size to humans, their immune system is quite similar to that of humans, and their long lifespan allows long-term efficacy and safety to be addressed.

### Example 2. Preliminary Pilot Study—Allogeneic Cells

A pilot study on 2 pediatric subjects from the HA sheep model described in Example 1. See Porada, et al., Phenotypic correction of hemophilia A in sheep by postnatal intraperitoneal transplantation of FVIII-expressing MSC. Exp Hematol, 2011. 39(12):1124-1135. During the first 3-5 months of life, both animals had received frequent, on-demand infusions of human FVIII (hFVIII) for multiple hematomas and chronic, progressive, debilitating hemarthroses of the leg joints which had resulted in severe defects in posture and gait, rendering them nearly immobile. Thus, for these subjects, FVIII was presented in the context of “danger signals”, which is known to trigger a robust host immune response to FVIII and other proteins.

Haploidentical MSC from the ram that had sired the two HA lambs were used for the therapy. The MSC were modified to introduce via transduction a B domain-deleted, wild-type porcine FVIII cDNA as described in Porada et al., Phenotypic correction of hemophilia A in sheep by postnatal intraperitoneal transplantation of FVIII-expressing MSC. Exp Hematol. 2011; 39(12): 1124-1135. MSC were simultaneously transduced with 2 lentivectors; one encoded eGFP for in vivo tracking of donor cells, and the second encoded an expression/secretion optimized porcine FVIII (pFVIII) transgene previously shown to be expressed/secreted from human cells at 10-100 times higher levels than hFVIII or sheep (ovine) FVIII (OFVIII). See Gangadharan et al., High-level expression of porcine factor VIII from genetically modified bone marrow-derived stem cells. Blood, 2006. 107(10): 3859-64; Doering et al., Directed Engineering of a High-expression Chimeric Transgene as a Strategy for Gene Therapy of Hemophilia A. Mol Ther, 2009. 17(7): 1145-54; Doering et al., Identification of porcine coagulation factor VIII domains responsible for high level expression via enhanced secretion. J Biol Chem, 2004. 279(8): 6546-52; Dooriss et al., Comparison of Factor VIII Transgenes Bioengineered for Improved Expression in Gene Therapy of Hemophilia A. Hum Gene Ther, 2009. 20(5): 465-78; Ide, L. M., et al., Hematopoietic stem-cell gene therapy of hemophilia A incorporating a porcine factor VIII transgene and nonmyeloablative conditioning regimens. Blood, 2007. 110(8): 2855-63; and Johnston et al., Generation of an optimized lentiviral vector encoding a high-expression factor VIII transgene for gene therapy of hemophilia A. Gene Ther, 2013. 20(6): 607-15.

FVIII/GFP-expressing MSC were then expanded and transplanted by IP injection (30×10⁶), in the absence of any preconditioning, into the first lamb. Following transplant, this animal's clinical picture improved dramatically, and he enjoyed an event-free clinical course, devoid of spontaneous bleeds, obviating the need for hFVIII infusions. Even more remarkably, the animal's existing hemarthroses resolved, his joints recovered fully, and he regained normal posture and gait, resuming a normal activity level. To the inventors' knowledge, this represents the first report of phenotypic correction of severe HA in a large animal model following transplantation of cells modified to express FVIII, and is the first time that reversal of chronic debilitating hemarthroses has been achieved in any setting.

Based on this remarkable clinical improvement, the modified MSC were transplanted into the second animal using an identical procedure, but a higher cell dose (125×10⁶). In similarity to the first animal, hemarthroses present in this second animal at the time of transplant resolved, he resumed normal activity shortly after transplantation, and became factor-independent.

Interestingly, despite the marked phenotypic improvement in both these animals, no circulating FVIII activity was detectable following the transplant, most likely due to the presence of high-titer inhibitors in these animals. These findings are remarkable, since despite the high titers of antibodies/inhibitors present in these animals, the transplanted allogeneic (haploidentical) MSC persisted and were not eliminated by the recipient's immune system, and the therapeutic effect of the treatment was maintained, i.e., the animals' symptoms of spontaneous joint bleeds, hematomas, and bleeding upon needle stick all improved.

### Example 3. Mechanistic Study-Autologous Cells

Twenty female HA carriers will be artificially inseminated (AI) via laparoscopy, as done in Example 2, with the support of the North Carolina State Theriogenology/Ruminant Medicine team. At 50-70 days of gestation (term: 150 days), amniotic fluid will be collected, and fetal cells from the amniotic fluid will be isolated, cultured, and expanded, using standard methods in our lab. Given the severe phenotype of these animals, we will perform a PCR-based RFLP (see Porada, C. D., et al., J Thromb Haemost, 2010. 8(2):276-85) to identify affected fetuses, allowing us to plan for their subsequent delivery. Following amniocentesis, the animals will be allowed to complete term. When the sheep have nearly completed gestation, the pregnant ewes carrying affected fetuses will be placed under close observation, and ewes will either be induced into labor using intramuscular dexamethasone for natural delivery, or the lambs will be delivered by Caesarian section, with clinical veterinarians assisting in either case. Both approaches have been used previously with success.

Affected lambs will be treated immediately with recombinant full-length or B-domain deleted ovine FVIII (OFVIII) produced as described in Zakas, P. M., et al., Development and characterization of recombinant ovine coagulation factor VIII. PLOS One, 2012. 7 (11): e49481. Although we have found that oFVIII is not a very high-expressing FVIII variant when compared to FVIII from other species, oFVIII protein for transfusion and an oFVIII transgene in the vectors are being used because the consensus in the hemophilia field is that the use of “same-species”FVIII is essential in preclinical gene therapy-based studies to accurately model the potential immune response in the clinical arena.

While human cells may be needed to perform definitive clinical studies, human cells are not appropriate for these mechanistic studies because using human cells would not allow us to address the critical issue of whether the use of autologous cells results in higher levels of long-term engraftment than we achieved in our pilot study with allogeneic cells, and whether the use of autologous cells may reduce the incidence of inhibitor formation. For this reason, sheep MSC will be used throughout this study.

It is our goal to treat HA with this MSC-based delivery system during the first 18 months of postnatal human life, since this is the time by which most HA patients would be diagnosed. Sheep develop much faster than humans, and are weaned at 60-90 days of age. We thus know this corresponds to the first 12-18 months for a human, so we will test the MSC-based treatment during the first 2-3 months of life in the sheep.

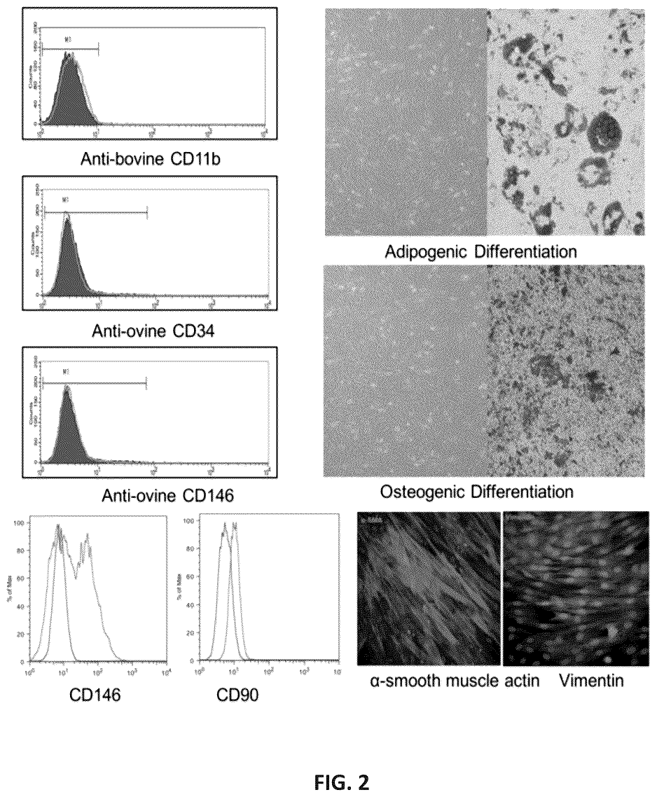

Starting at birth, HA lambs will be treated prophylactically 2-3 times per week with recombinant oFVIII. At 4-5 weeks of age, we will collect bone marrow (under oFVIII coverage), and isolate MSC from each affected lamb, as we have done previously. These methods enable us to successfully establish primary sheep MSC that are phenotypically and functionally similar to their human counterparts; these sheep MSC are devoid of hematopoietic cells (they lack CD11b, CD34, and CD45), but they express the MSC markers CD146 and CD90. See FIG. 2. Anti-ovine antibodies to other antigens routinely used to identify MSC, such as CD44, CD105, and CD73, are not commercially available. Immunofluorescence microscopy demonstrated expression of vimentin and α-smooth muscle actin, known MSC cytoskeleton proteins, and we verified the ability of these sheep MSC were able to differentiate into adipocytes (by Oil-red-o staining) and osteocytes (by calcium deposition and alkaline phosphatase). See FIG. 2.

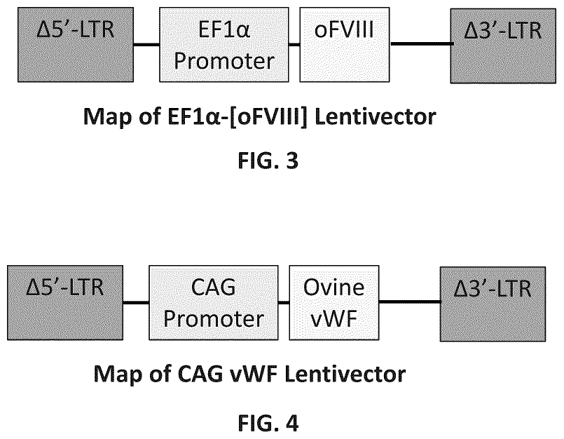

We will then subject the isolated MSC to 2-3 rounds of transduction with either the EF1α-[OFVIII] lentivirus vector (FIG. 3) alone, or simultaneously with the EF1α-[OFVIII] lentivirus vector and the CAG-[vWF] lentivirus vector (FIG. 4). See De Meyer, S. F., et al., Phenotypic correction of von Willebrand disease type 3 blood-derived endothelial cells with lentiviral vectors expressing von Willebrand factor. Blood, 2006. 107 (12): 4728-36. The EF1α-[OFVIII] lentivirus vector contains a B-domain deleted oFVIII gene having the polynucleotide sequence set forth in SEQ ID NO: 33. The CAG-[vWF] lentiviral vector contains a truncated vWF having the polynucleotide sequence set forth in SEQ ID NO: 29. From the pilot study described in Example 2, we know that 2-3 rounds of simultaneous exposure to two different lentivirus vectors results in transduction of 90-95% of sheep MSC with both vectors. The lentivirus vectors for these studies both contain the 3′-modified LTR to produce SIN lentivirus vectors, and the constitutive EF1α promoter will drive FVIII expression. Due to packaging constraints, it is not possible to utilize the EF1α promoter in the vWF lentivirus vector. Prior studies have established the ability of vWF to be packaged within a lentivirus vector and expressed at high levels. See De Meyer, S. F., et al., 2006 (above). However, if any difficulties arise packaging vWF-encoding lentivirus vectors, we will utilize truncated vWF cassettes as described in this disclosure, or switch to foamy virus vectors, as they possess a much larger packaging capacity.

We are including a group in which autologous MSC are transduced with vectors encoding both OFVIII and vWF (ovine vWF; GI: 426227037) for two reasons: 1) binding to vWF stabilizes FVIII and prolongs its half-life and, thus, delivery of MSC secreting FVIII complexed with vWF may produce a more pronounced therapeutic effect; and 2) vWF may reduce the immunogenicity of exogenously administered FVIII by preventing both its uptake and presentation by dendritic cells, and its recognition by immune effector cells. We predict that delivering the two proteins in the same vector will result in the release of FVIII: vWF as a complex from the transduced MSC. We will confirm co-localization/complex formation of vector-derived oFVIII and vWF in transduced MSC populations by confocal microscopy prior to performing the proposed transplants. Although MSC do not endogenously produce any vWF, we will add a 6-His tag (SEQ ID NO:32) to the vWF transgene, making it readily distinguishable from any trace endogenous vWF for these in vitro studies.

Following transduction, the FVIII-expressing MSC will be expanded until the animals have reached 2-3 months of age, at which point the MSC be transplanted autologously into their respective donor HA lamb, via IP injection under ultrasound guidance, with no preconditioning (as in our pilot study [23]), using a dose of 5-10×10⁶ cells/kg. An aliquot of each cell type will be reserved to determine vector copy number by qPCR and to perform integration site analysis by LM-PCR. Methods described in Porada, C. D., et al., Phenotypic correction of hemophilia A in sheep by postnatal intraperitoneal transplantation of FVIII-expressing MSC. Exp Hematol, 2011. 39(12): 1124-1135 (qPCR) and Russo-Carbolante, E. M., et al., Integration pattern of HIV-1 based lentiviral vector carrying recombinant coagulation factor VIII in Sk-Hep and 293T cells. Biotechnol Lett, 2011. 33 (1): 23-31 and Tellez, J., et al., High Incidence of Vector Integration Near Cancer-Related Genes within Primitive Hematopoietic Stem Cells (HSC) After Fetal Gene Transfer with γ-Retroviral Vectors. Molecular Therapy, 2010. 18(Suppl. 1): p. S331 (LM-PCR). Two experimental groups will be included: 1) autologous MSC transduced with the EF1α-[OFVIII] lentivector (n=2-3 HA lambs); and 2) autologous MSC transduced with both the EF1α-[OFVIII] and CAG-vWF lentivectors (n=2-3 HA lambs).

Following transplantation, prophylactic oFVIII infusions will be discontinued, and any benefit as a result of this MSC-based approach should be readily apparent, given the severe, life-threatening phenotype of these animals. The sheep will be continually monitored for bleeds, and platelet-deficient plasma will be collected monthly until at least 1.5 years of age for coagulation assays, to quantify the plasma levels of oFVIII by chromogenic assay and/or ELISA. The formation/presence of inhibitors will be assessed at each time point by performing the Nijmegen modification of the Bethesda assay (as described in Verbruggen, B., et al., The Nijmegen modification of the Bethesda assay for factor VIII: C inhibitors: improved specificity and reliability. Thromb Haemost, 1995. 73 (2): 247-51) and a commercially available kit (Technoclone/DiaPharma Group, Inc.) on an aliquot of plasma collected from the animals. Once we have obtained these values, we will compare the HA lambs that received MSC transduced with the lentivector encoding oFVIII alone to those that received MSC transduced with both the OFVIII and vWF lentivectors, and compare each of these to the historic values from untransplanted HA sheep, and to a reference panel of normal unaffected males. These studies will allow us to: 1) establish what levels of vector-encoded oFVIII are expressed as a result of this postnatal approach; 2) determine the duration of the therapeutic effect of this approach; 3) assess whether using autologous MSC and a lentivector that lacks eGFP avoids the inhibitors seen in the pilot study of Example 2; and 4) establish whether including vWF improves the therapeutic effect of this MSC-based treatment and/or reduces the incidence/titer of inhibitor formation.

At 1.5 years of age (or sooner, if we see that FVIII levels are dropping), the HA lambs will be euthanized, and all major organs will be harvested. All tissues will be fixed in 4% paraformaldehyde, processed through a sucrose gradient, embedded and frozen in OCT, and sectioned at 5 μm. 8-10 slides/tissue will be stained with an antibody specific to oFVIII and analyzed/quantitated by confocal microscopy for the presence of engrafted MSC to precisely determine the levels and localization (parenchymal vs. perivascular) of engrafted cells that are expressing FVIII, and are therefore providing therapeutic benefit. We will also collect plasma from each of these recipients at the time of euthanasia, and quantitate the circulating levels of vector-derived oFVIII in these sheep using an ELISA specific for oFVIII, correlating levels and patterns of engraftment with circulating FVIII levels. Based on ease of access to the circulation, we hypothesize that maximal plasma FVIII levels will be obtained when MSC lodge in perivascular regions of the engrafted tissues.

While confocal analysis should provide us with a fairly accurate estimate of the levels of oFVIII+MSC within each tissue, the slides selected for quantitation may or may not be representative of the engraftment levels within the organ as a whole. Therefore, we will also use an ELISA to precisely quantitate the amount of oFVIII within tissue homogenates. A standard curve will be created with known numbers of oFVIII+MSC, thereby establishing how much oFVIII is present on a per-cell basis. Protein extracts will then be prepared from the tissues from each animal and analyzed using this ELISA, comparing the tissue values to that of the standard curve, to precisely quantitate the number of MSC that have engrafted within each tissue and are expressing oFVIII. We will then compare the levels of donor MSC in each tissue with the resultant plasma FVIII levels to determine in which tissues engraftment produces the highest circulating levels of FVIII.

### Example 4. Study Assessing Treatment in Subjects with Pre-Existing Inhibitors

The findings of the study described in Example 3 will be used to determine the ability of this autologous cell-based approach to mediate clinical/phenotypic improvement in recipients with pre-existing inhibitors as a result of on-demand FVIII treatment.

Three to four HA lambs will be treated on-demand with oFVIII beginning at birth. We expect, based on the pilot study of Example 2, that treating on-demand with FVIII products will result in the formation of inhibitors in almost all HA sheep by 4-5 months of age. We will collect bone marrow and isolate MSC at 4-5 weeks of age, and transduce these cells with either the EF1α-[OFVIII] lentivector alone, or with both the EF1α-[OFVIII] and CAG-vWF lentivectors (depending which approach yields the best outcome in the preceding studies), and expand the transduced cells to obtain adequate numbers for transplant.

Beginning at birth, we will draw blood from these animals every other week to obtain plasma and perform the Nijmegen modification of the Bethesda assay (as above) (Technoclone/DiaPharma Group, Inc.) to assess the development of inhibitors. Once inhibitors have developed, we will transplant the transduced autologous MSC into each animal by ultrasound-guided IP injection, as in Exp. Set #1.1, at a dose of 5-10×10⁶ cells/kg.

Following transplantation, we will analyze the animals as detailed in Example 3, continually monitoring them for bleeds, and collecting platelet-deficient plasma monthly until at least 1 year of age for coagulation assays, to quantify the plasma levels of oFVIII by chromogenic assay and/or ELISA, and to quantitate the levels of inhibitors present, to ascertain whether the MSC-based treatment impacts upon the levels of the pre-existing inhibitors. We will then compare the results obtained with these HA lambs with pre-existing inhibitors to those in Example 3 in which the HA lambs lacked inhibitors at the time of MSC infusion.

### Example 5. First Line Therapy Study

We hypothesize that previously untreated patients (PUPs) represent the ideal group to initially target with this MSC-based treatment, because their immune systems are completely naïve to exogenous FVIII, and will be exposed to it for the first time when it is released by the transplanted MSC; we anticipate this will reduce/eliminate the risk of inhibitor formation in this population.

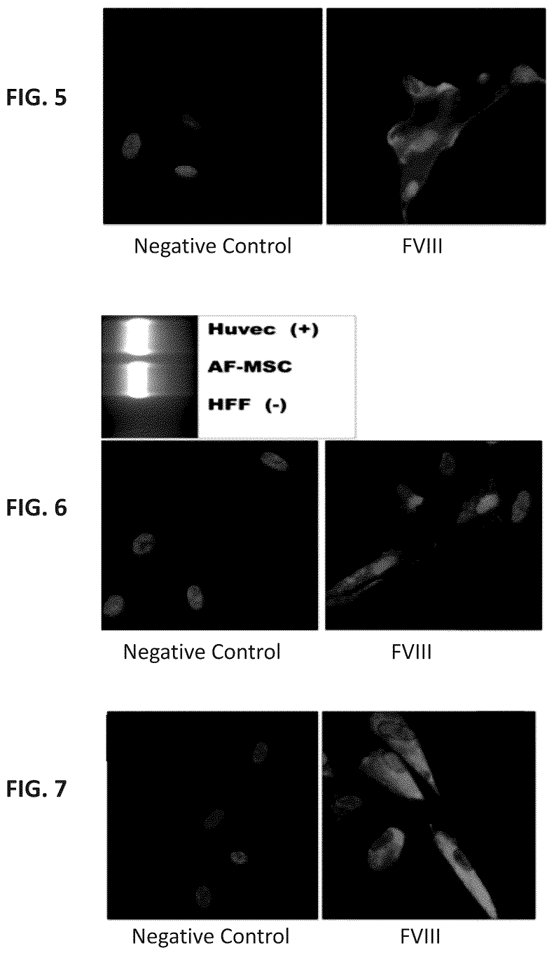

In families with no prior history, HA is normally diagnosed during the first 18 months of life, after the child exhibits abnormal bruising/bleeding after a minor trauma. In families with a history of HA, diagnosis can be made at birth, or even prior to birth [148-158]. Regardless of when diagnosis is made, however, it would not be possible to collect bone marrow MSC from these patients without treating with factor to prevent hemorrhage during the procedure. This same issue exists with the HA sheep, since they present with a severe phenotype and spontaneous bleeding from birth. However, in similarity to patients with a family history of HA, the affected sheep can be diagnosed in utero by amniocentesis (as can be done in human patients), making it possible to collect autologous cells from the amniotic fluid part way through gestation, transduce these cells, expand them, and have them ready to transplant as the first-line therapy at birth, or shortly thereafter. We recently found that MSC-like cells present within the amniotic fluid, “AF-MSC”, are readily transduced with lentivirus vectors, they endogenously produce low levels of FVIII (FIG. 5) and vWF (FIG. 6), and they express very high levels of FVIII following lentivector transduction (FIG. 7). These findings suggest that amniotic fluid can replace marrow as a source of autologous MSC for delivering a FVIII transgene, enabling us to test the MSC-based treatment's efficacy as first-line therapy in PUPs.

To test the efficacy of the MSC-based treatment in PUPs, HA carrier ewes will be bred or artificially inseminated as detailed in Example 3. At 50-60 days of gestation (term: 150 days), amniotic fluid will be collected, AF-MSC isolated, and a PCR-based RFLP performed to detect the HA mutation, as detailed in Example 3. AF-MSC from the affected fetuses will then be subjected to 2-3 rounds of transduction with the EF1α-[OFVIII] comprising the polynucleotide sequence set forth in SEQ ID NO: 33 (FIG. 3) and expanded. At near term, labor will be induced in the ewes with affected lambs, or a C-section performed to deliver the lambs. Immediately after birth, the HA lamb “PUPs” (n=3-4) will be injected IP with the transduced autologous AF-MSC at a dose of 5-10×10⁶ cells/kg, as described for the BM-MSC in Example 3.

Following transplantation, we will analyze the animals as detailed in Example 3 and 4, continually monitoring them for bleeds, and collecting platelet-deficient plasma monthly until at least 1 year of age for: 1) coagulation assays; 2) to quantify the plasma levels of oFVIII by chromogenic assay and/or ELISA; and 3) to perform the Nijmegen modified Bethesda assay to assess the development of inhibitors. We will then compare the results obtained by using this MSC-based treatment as a first-line therapy in these HA lamb “PUPs” to the results obtained in the HA lambs treated prophylactically (Example 3) and to those treated on-demand (Example 4) prior to MSC infusion.

### Example 6. Study to Assess Induction of Immune Tolerance to Factor VIII

We hypothesize that the continued delivery of FVIII to the circulation by the lentivector-modified MSC can serve as a much-needed novel method of inducing immune tolerance to FVIII.

The aim of this study is to test the ability of this MSC-based approach as a novel method of inducing immune tolerance through the continued delivery of FVIII to the circulation by the genetically-modified MSC. To accomplish this objective, 2-3 HA lambs (more will be added if initial data are not clear-cut) will be treated on-demand with oFVIII, beginning at birth, as we know that treating on-demand with FVIII products results in the formation of inhibitors in almost all HA sheep by 4-5 months of age. As in Example 4, we will isolate BM-MSC at 4-5 weeks of age, and transduce these cells with both the EF1α-[OFVIII] and CAG-vWF lentivectors, as clinical data indicate that the inclusion/presence of vWF may facilitate ITI [123, 159, 160]. The transduced cells will then be expanded to obtain adequate numbers for subsequent transplant.

Beginning at birth, we will draw blood from these animals every other week to obtain plasma and perform the Nijmegen modified Bethesda assay (as above) to assess inhibitor induction. Once inhibitors have developed, we will transplant transduced autologous MSC, at a dose of 10⁷ cells/kg, into the peritoneal cavity of each animal, as in Example 3. This procedure will be repeated each 4-5 days until we observe a drop in inhibitor titer (as detailed below); a maximum of 10 infusions will be given initially.

Following transplantation, we will analyze the animals as in Example 3, continually monitoring for bleeds (as the repeated MSC-based treatment should produce clinical/phenotypic improvement), and collecting platelet deficient plasma bi-weekly for ≥3 months, to perform coagulation assays, to quantify the oFVIII plasma levels by chromogenic assay and/or ELISA, and to quantitate the levels of inhibitors present, to ascertain whether the repeated infusion of MSC expressing high levels of FVIII can break the existing inhibitors and induce tolerance to FVIII. To further confirm that this cell-based ITI has overcome the existing inhibitors, we will assess the restoration of normal FVIII pharmacokinetics, using well-established methodology (plasma FVIII recovery ≥66% of expected and a ≥6 h half-life, determined following a 72-hour FVIII-exposure-free period).

### Example 7. Comparison of Recombinant Factor VIII Infusion Therapy with MSC-Based Tolerance Induction

One of the only clinical options for HA patients who develop inhibitors is immune tolerance induction (ITI), which involves the long—term administration of high doses of FVIII protein. ITI is extremely expensive, is only effective in a percentage of patients with inhibitors, and the mechanism for its success is unknown. To-date, no preclinical HA model has been used to study and/or optimize ITI. Given the high incidence of inhibitor formation in the HA sheep and their lack of any cross-reactive material, they represent an excellent model in which to investigate ITI. We propose to perform a head-to-head comparison of traditional ITI, using repeated high-dose recombinant oFVIII to the MSC-based ITI protocol developed/tested in Example 6.

To achieve these goals, 2-3 HA lambs will be treated on-demand with oFVIII, beginning at birth, as described in Example 6, until inhibitors develop. We will then commence a clinically employed, protein-based ITI regimen, infusing the inhibitor animals with a dose of 100 IU/kg/day for 3 months (as described in Oldenburg, J., et al., Primary and rescue immune tolerance induction in children and adults: a multicentre international study with a VWF-containing plasma-derived FVIII concentrate. Haemophilia, 2014. 20(1):83-91). During the course of this ITI protocol, we will analyze the animals as detailed in Example 6, collecting platelet-deficient plasma weekly, to: 1) perform coagulation assays; 2) quantify the plasma levels of oFVIII by chromogenic assay and/or ELISA; and 3) quantitate the levels of inhibitors present, to assess the ability of the protein-based ITI to break existing inhibitors, and define the kinetics with which this happens. To further confirm that ITI has overcome existing inhibitors, we will assess the restoration of normal FVIII pharmacokinetics, as detailed above. The success rate and kinetics of tolerance induction with the protein-based ITI will be compared to those of the cell-based protocol in Example 6, to determine whether the cell-based method is a viable alternative to the time consuming and expensive protein-based method that represents the current state-of-the-art in clinical ITI.

### Example 8. Transduction Efficiency with Different Vectors

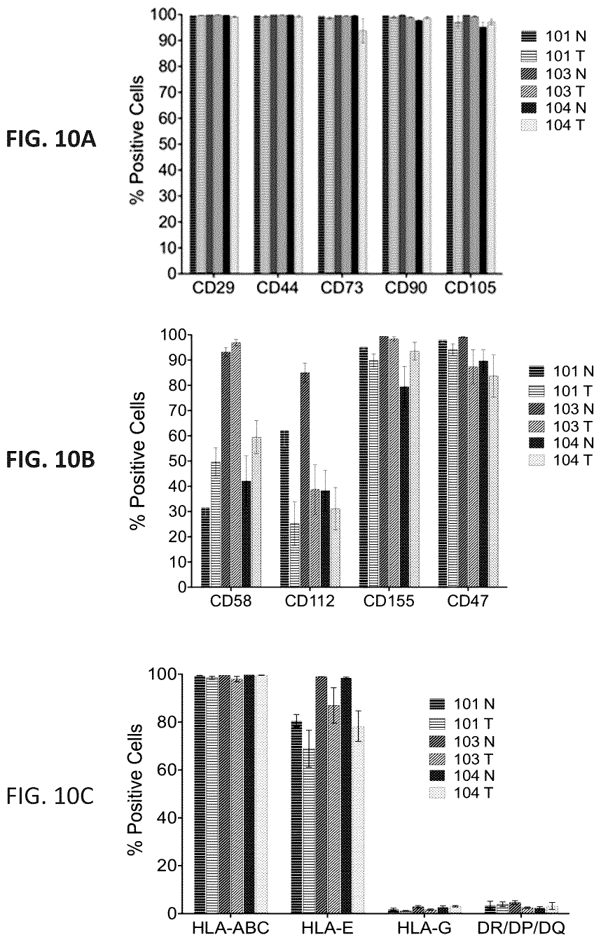

The transduction efficiency, FVIII production, and FVIII secretion from human PLC following transduction at an identical multiplicity of infection (MOI) of 7.5 with an identical lentiviral vector (LV) encoding one of the following four different FVIII transgenes: (1) a bioengineered human-porcine hybrid FVIII (ET3) having the polynucleotide sequence set forth in SEQ ID NO: 11; (2) a liver codon-optimized ET3 (lcoET3) having the polynucleotide sequence set forth in SEQ ID NO: 10; (3) a liver codon-optimized human FVIII (IcoHSQ) having the polynucleotide sequence set forth in SEQ ID NO: 14; and (4) a myeloid-codon optimized ET3 (mcoET3) having the polynucleotide sequence set forth in SEQ ID NO: 12 were compared. Brown et al. (2018) Mol. Ther. Methods Clin. Dev. 9:57-69, demonstrated that vectors encoding FVIII, when codon-optimized to the target cells, or tissue, result in a dramatically increase FVIII expression of functional FVIII. Following transduction, PLCs were analyzed by flow cytometry and confocal microscopy to measure transduction efficiency and FVIII production. Conditioned media of PLCs were assayed by aPTT to quantitate FVIII activity. Analysis of the culture supernatants by aPTT demonstrated FVIII activity was readily detectable in supernatants of all transduced cells lines. It also revealed marked differences in the secretion of functional FVIII following transduction with each of these vectors. Specifically, PLCs transduced with mcoET3 (SEQ ID NO: 12), ET3 (SEQ ID NO: 11), IcoET3 (SEQ ID NO: 10), and lcoHSQ (SEQ ID NO: 14) LV secreted 25±9, 19±8, 11±2, and 1±0.1 IU of FVIII/24 h/10⁶ cells, respectively (FIG. 10). PLC population doubling time was not affected by transduction with any of the vectors; nor were phenotype or expression of signaling molecules involved in innate immunity. Importantly, at passage 3 following transduction with any of the 4 lentiviral vectors, PLCs continued to produce and secrete FVIII at similar levels to those observed shortly after transduction, demonstrating stable vector integration and durability/longevity of FVIII expression. The relative levels of FVIII expression by PLCs following transduction with each lentiviral vector were also assessed by immunofluorescence microscopy with an antibody specific to a region of FVIII that is conserved in all 4 FVIII transgenes. These analyses confirmed the results of the aPTT analyses on the supernatants from these cells, with mcoET3-PLC exhibiting the brightest/highest intensity staining for FVIII, followed by PLC transduced with ET3 (SEQ ID NO: 11), then those transduced with lcoET3 (SEQ ID NO: 10) and with IcoHSQ (SEQ ID NO: 14) (data not shown).

The gene transfer efficiency of these gene-modified cells was assessed by determining the final proviral/vector copy number (VCN) using a commercially available qPCR-based kit (Lenti-X Provirus Quantitation Kit, Takara Bio USA, Inc., Mountain View, CA). To ensure that only integrated copies were detected by the assay, qPCR for VCN was performed in PLCs that had been passaged at least three times after transduction. After transducing the cells at the same MOI (7.5) with each lentiviral vector, the VCNs for mcoET3-PLC, IcoHSQ-PLC, IcoET3-PLC, and ET3-PLC were all around 1.

### Example 9. Optimizing Factor VIII Expression in Placental Cells

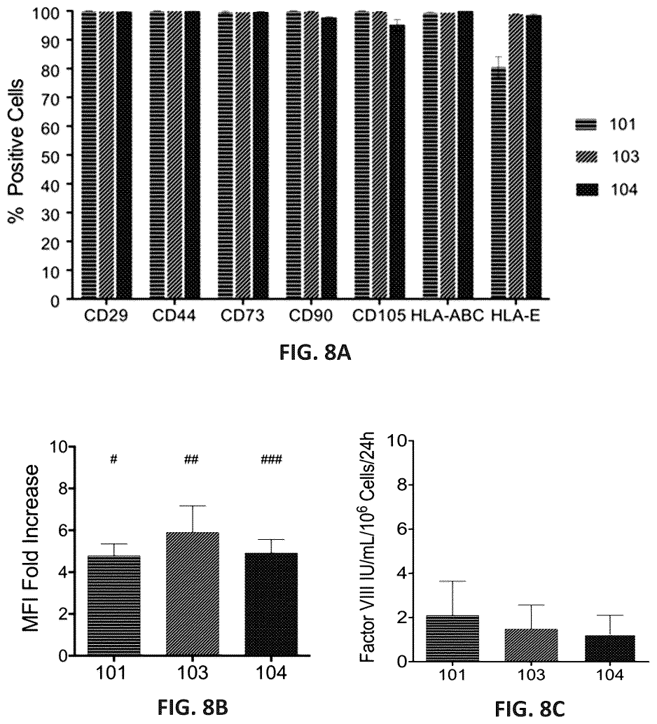

The aim of this study was to investigate the suitability of placental cells (PLC) as cellular delivery vehicles for FVIII. The expression of phenotypical markers was determined in three different master cells banks (101, 103, and 104) of placental cells (PLCs), each of which was derived from a different human donor by the Regenerative Medicine Clinical Core (RMCC) at WFIRM following GMP-compliant standard operating procedures (SOPs) established by the RMCC for PLC. Expression of CD29, CD44, CD73, CD90, CD105, HLA-ABC, HLA-E, CD31, CD34, CD35, CD144, HLA-G, HLA-DR/DP/DQ, and ABO blood group were determined using flow cytometric analysis. These markers were selected to confirm that the PLC isolated possessed a phenotype characteristic of MSC from other tissues (CD29, CD44, CD73, CD90, CD105), to assess their potential for stimulating an immune response upon transplantation (HLA-ABC, HLA-E, CD35, CD144, HLA-G, HLA-DR/DP/DQ, and ABO blood group), and to discern whether they expressed markers indicative of endothelial properties (CD31, CD34). No statistically significant differences (p<0.05) were found in expression of phenotypic markers between PLCs derived from three different master cell banks (101, 103, and 104). PLCs from each of the master cell banks expressed CD29, CD44, CD73, CD90, CD 105, HLA-ABC, and HLA-E (FIG. 8A); had negligible amounts (<1%) of CD31, CD34, CD35, CD144, HLA-G, and HLA-DR/DP/DQ (data not shown); and were devoid of ABO blood group (data not shown). Collectively, these findings support the conclusion that PLC are an MSC-like population and that they should exhibit minimal immunogenicity upon transplantation.

PLCs derived from each of the master cell banks [MCBs] (101, 103, and 104) were assessed for their ability to express FVIII protein constitutively. Immunofluorescence microscopy with a primary antibody specific to hFVIIIc and a fluorochrome-conjugated secondary antibody and flow cytometric analysis with fluorochrome-conjugated antibodies to were used to determine the levels of constitutively expressed FVIII protein and define the phenptype of the PLCs, respectively. As shown in FIG. 8A, these cells expressed markers characteristic of MSC from bone marrow and other tissues. All three MCBs endogenously expressed detectable amounts of FVIII by immunofluorescence microscopy, and MCB 103 expressed the highest levels, as indicated by the brightest fluorescence intensity (data not shown). The fold increase of FVIII expression over isotype control for PLCs derived from each of the MCBs is presented as relative mean fluorescence intensity (MFI) as shown in FIG. 8B.

The activated partial thromboplastin time (aPTT or PTT) assay is a functional measure of the intrinsic and common pathways of the coagulation cascade (i.e. it characterizes blood coagulation). The aPTT assay was used to quantitate levels of functional FVIII secreted by PLCs. PLCs were plated at the same density and cultured for 24 hours in Phenol Red-free alpha-MEM AmnioMax Complete Medium (ThermoFisher Scientific, Raleigh, NC). Supernatants were collected and the number of cells present counted, and then the levels of secreted functional FVIII were measured by the Clinical Hematology Laboratory at Wake Forest Baptist Health using a commercial aPTT assay. Levels of FVIII were normalized by adjusting to account for the number of cells present at the time of supernatant collection, and expressing FVIII activity on a per cell basis. The data from this analysis is shown in FIG. 8C

FVIII mRNA levels in the PLCs derived from three different master cell banks (101, 103, and 104) were evaluated by qPCR using primers specific to human FVIII. Relative expression of endogenous mRNA for FVIII was calculated by comparing the threshold cycle (CT) value for FVIII with the CT of each master cell bank's respective internal reference gene, GAPDH. The relative expression of endogenous FVIII mRNA was 0.01±0.0005, 0.075±0.007, and 0.011±0.0002, for PLCs 101, 103, and 104, respectively.

### Example 10. Suitability of PLCs as a Transgenic FVIII Production Platform

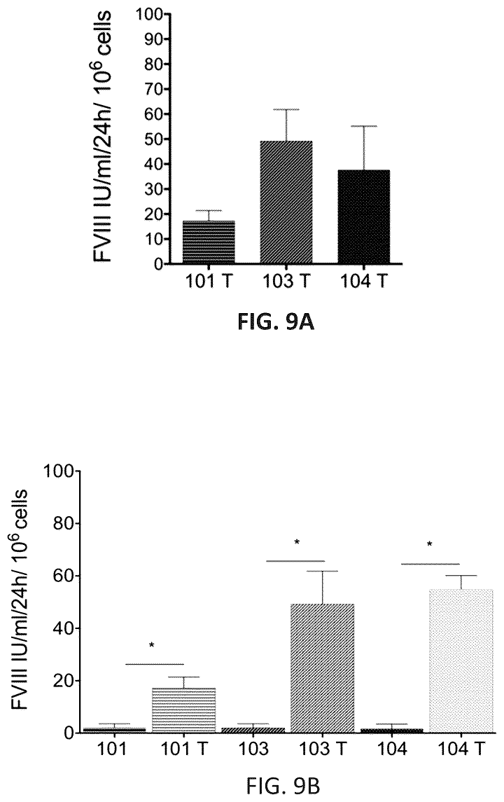

PLC 101, 103, and 104 were transduced at the same MOI (7.5) using a lentiviral vector-(LV) encoding mcoET3 (mcoET3-PLC) as described in Example 4 above. Vector copy number (VCN) was determined, as described in detail above. The VCN was found to be similar between the three different PLC MCBs (0.71-0.75). After transduction, the relative levels of expression of FVIII by the 3 MCB PLCs were assessed by immunofluorescence microscopy after staining with a primary antibody specific to hFVIIIc and a fluorochrome-conjugated secondary antibody. All 3 MCBs expressed high levels of FVIII after transduction with the mcoET3 lentiviral vector, but MCB 103 exhibited the highest levels of FVIII protein, as evidence by the brightest/highest fluorescence intensity (data not shown). The secretion of FVIII was determined using aPTT performed on 24-hour culture supernatants harvested from PLCs that were plated at the same density and normalized for the number of cells present at the time of the supernatant collection, as described in detail in the preceding paragraphs (FIG. 9A). Levels of FVIII in the culture supernatant increased significantly (p<0.05) in PLCs derived from all 3 MCBs (101, 103, and 104) when compared with respective non-transduced PLC counterparts (FIG. 9B). No significant differences were found between the different transduced cells.

The effect of transduction of PLCs with LV encoding mcoET3 on phenotype or molecules involved in immunity was assessed. Expression of CD29, CD44, CD73, CD90, CD 105, CD58, CD112, CD155, CD47, HLA-ABC, HLA-E, HLA-G, and HLA-DR/DP/DQ, were determined by flow cytometric analysis, as described above. No statistically significant differences (p<0.05) were found between transduced and non-transduced cells (FIGS. 8A-8C). Additionally, PLC population doubling time was not affected by transduction (data not shown). Both non-transduced and transduced PLCs expressed CD47, a molecule involved in immune evasion (FIG. 8B). Transduced cells did not significantly upregulate the expression of HLA-DR/DP/DQ (FIG. 8C).

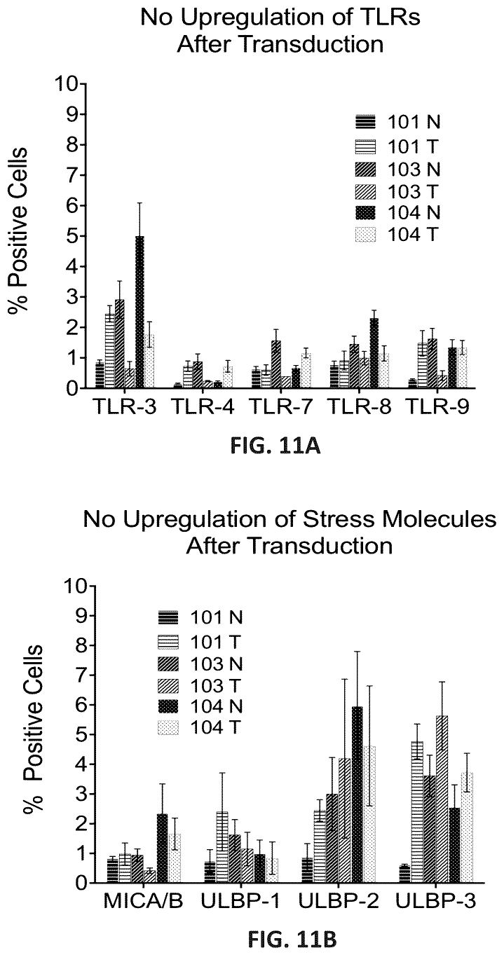

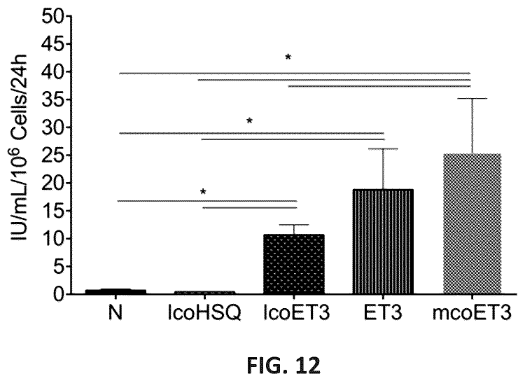

To further examine whether transduction of the PLC with the mcoET3 lentiviral vector had the potential to alter the immunogenicity of these cells, we examined the levels of expression of various Toll-like Receptors (TLRs) on the PLC prior to and following transduction, as these molecules play a key role in innate immunity, and their upregulation could potentially trigger an immune response to the transduced cells upon transplantation. To address this possibility, the effect of PLC transduction with the mcoET3 lentiviral vector on TLR-3, TLR-4, TLR-7, TLR-8, and TLR-9 expression was assessed. TLR expression on transduced (t) and non-transduced (n) PLCs (101, 103, and 104) was determined using flow cytometric analysis. No significant differences in expression of TLR molecules was detected in the PLC populations. As shown in FIG. 11A, there was no difference in the levels of expression of any of these TLRs in transduced vs. non-transduced PLCs, confirming that transduction of these 3 MCB PLCs with the mcoET3 lentiviral vector did not lead to upregulation of any of these immune-stimulating molecules.

In order to evaluate the demands of PLC transduction with mcoET3 and increased Factor VIII expression on the secretory and endoplasmic reticulum pathways, expression of stress molecules MICA/B, ULBP-1, ULBP-2, and ULBP-3 was determined in transduced (t) and non-transduced (n) PLCs (101, 103, and 104). Flow cytometric analysis demonstrated that no significant expression or alteration/upregulation of MICA/B or ULBP-1 were found before or after transduction with mcoET3 lentiviral vector (FIG. 11B).

The production of interferon-gamma (IFN-γ) by mcoET3 transduced and non-transduced PLCs was measured using a high-sensitivity ELISA (assay range: 0.16-10.0 μg/mL). PLCs were cultured for 24 hours in AmnioMax Complete Medium (ThermoFisher). Supernatants were collected and IFN-γ production was determined. No IFN-γ was detected in any of the culture supernatants of mcoET3-transduced or non-transduced PLCs (data not shown).

All features of the described systems are applicable to the described methods mutatis mutandis, and vice versa.

All patents, patent publications, patent applications, journal articles, books, technical references, and the like discussed in the instant disclosure are incorporated herein by reference in their entirety for all purposes.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments, such substitution is considered within the scope of the disclosure.

The examples presented herein are intended to illustrate potential and specific implementations of the invention. It can be appreciated that the examples are intended primarily for purposes of illustration for those skilled in the art. There may be variations to these diagrams or the operations described herein without departing from the spirit of the invention. For instance, in certain cases, method steps or operations may be performed or executed in differing order, or operations may be added, deleted or modified.

Different arrangements of the components depicted in the drawings or described above, as well as components and steps not shown or described are possible. Similarly, some features and sub-combinations are useful and may be employed without reference to other features and sub-combinations. Aspects and embodiments of the invention have been described for illustrative and not restrictive purposes, and alternative embodiments will become apparent to readers of this patent. Accordingly, the present invention is not limited to the embodiments described above or depicted in the drawings, and various embodiments and modifications can be made without departing from the scope of the claims below.

While exemplary embodiments have been described in some detail, by way of example and for clarity of understanding, those of skill in the art will recognize that a variety of modification, adaptations, and changes may be employed. Hence, the scope of the present invention should be limited solely by the claims.

What is claimed is:

1. A method of treating a subject diagnosed with hemophilia A, comprising:
(a) modifying mesenchymal stem/stromal cells (MSC) to express high levels of Factor VIII protein thereby generating modified MSC, the MSC comprising bone-marrow MSC isolated from the subject;
(b) generating an expanded modified MSC population by in vitro culturing the modified MSC; and
(c) injecting MSC from the expanded modified MSC population into the subject.

2. The method of claim 1, wherein isolating the MSC comprises isolating cells that express at least one of Stro-1 or CD146.

3. The method of claim 1, wherein the subject has received prior treatment with exogenous Factor VIII and has developed an inhibitory immune response that diminishes the effectiveness of the exogenous Factor VIII treatment.

4. A method of treating a subject prenatally diagnosed as having hemophilia A, comprising:
(a) modifying mesenchymal stem/stromal cells (MSC) to express high levels of Factor VIII protein thereby generating modified MSC, the MSC comprising MSC isolated from at least one of amniotic fluid, placental tissue, or umbilical cord tissue obtained at the time of the subject's birth or prenatally from the subject's mother;
(b) generating an expanded modified MSC population by in vitro culturing the modified MSC; and
(c) injecting MSC from the expanded modified MSC population into the subject.

5. The method of claim 4, wherein isolating the MSC comprises isolating cells that express c-kit.

6. The method of claim 4, wherein the isolated MSC express c-kit, CD34, CD90, and CD133.

7. The method of any one of the preceding claims, wherein the MSC isolated from the subject are modified to express high levels of von Willebrand factor (vWF) protein.

8. The method of any one of the preceding claims, wherein the Factor VIII protein comprises at least one of human Factor VIII protein sequence, ovine Factor VIII protein sequence, or porcine Factor VIII protein sequence.

9. The method of any one of the preceding claims, wherein the Factor VIII protein comprises one or more modifications that increase protein expression, protein stability, or both.

10. The method of any one of claims 7-9, wherein the vWF protein comprises at least one of human vFW protein sequence, ovine Factor VIII protein sequence, or porcine vWF protein sequence.

11. The method of any one of claims 7-10, wherein the vWF protein comprises one or more modifications that increase protein expression, protein stability, or both.

12. The method of any one of the preceding claims, wherein the MSC are modified to express high levels of Factor VIII protein via introduction into the MSC of a viral vector comprising a Factor VIII gene sequence operatively linked to a constitutively active promoter.

13. The method of any one of claims 1-11, wherein the MSC are modified to express high levels of the Factor VIII protein via gene editing of an endogenous Factor VIII gene sequence of the MSC, wherein the gene editing introduces one or more modifications to an endogenous Factor VIII gene sequence that increase protein expression, protein stability, or both, of the Factor VIII protein.

14. The method of any one of claims 7-13, wherein the MSC are modified to express high levels of vWF protein via introduction into the MSC of a viral vector comprising a vWF gene sequence operatively linked to a constitutively active promoter.

15. The method of claim 14, wherein the MSC are modified to express high levels of Factor VIII protein and vWF protein via introduction into the MSC of a viral vector comprising a Factor VIII gene sequence operatively linked to a constitutively active promoter and a vWF gene sequence operatively linked to a constitutively active promoter.

16. The method of claim 15, wherein the Factor VIII gene sequence and the vWF gene sequence are operatively linked to the same constitutively active promoter.

17. The method of any one of claims 1-13, wherein the MSC are modified to express high levels of the vWF protein via gene editing of an endogenous vWF gene sequence of the MSC, wherein the gene editing introduces one or more modifications to the endogenous vWF gene sequence that increase protein expression, protein stability, or both, of the vWF protein.

18. The method of any of the preceding claims, wherein the MSC from the expanded modified MSC population are injected into the subject via at least one of intraperitoneal injection, intravenous injection, or intra-articular injection.

19. The method of any one of the preceding claims, wherein the expanded modified MSC population are injected into the subject at least once, at least twice, or at least three times.

20. The method of any one of the preceding claims, wherein the expanded modified MSC population are injected into the subject in an amount of about 10⁷ to about 10⁹ MSC per kilogram weight of the subject.
